(19) United States
(12) Patent Application Publication
(10) Pub. No.: US 2026/0284275 A1
(43) Pub. Date: Sep. 24, 2026

(54) NANOGEL COMPRISING BIOACTIVE MOLECULES, THERAPEUTIC MOLECULES OR DRUGS TOGETHER WITH AN AMPHIPHILIC MOLECULE

(71) Applicant: CM4CURE S.A., Liege (BE)

(72) Inventors: Cécile OURY, Liege (BE); Patrizio LANCELLOTTI, Liege (BE); Abdelhafid AQIL, Liege (BE); Bartosz DITKOWSKI, Liege (BE)

(21) Appl. No.: 19/164,517

(22) PCT Filed: Feb. 14, 2024
(86) PCT No.: PCT/EP2024/053778

§ 371 Date: Sep. 11, 2025

(30) Foreign Application Priority Data
Mar. 15, 2023 (EP) 23162106.1
Mar. 28, 2023 (EP) 23164834.6

(51) Int. Cl. A61L 29/04; A61L 29/08; A61L 29/14; A61L 29/16

(52) U.S. Cl. CPC A61L 29/041; A61L 29/085; A61L 29/145; A61L 29/16; A61L 2300/204; A61L 2300/206; A61L 2300/406; A61L 2400/12; A61L 2420/02; A61L 2420/08

(57) ABSTRACT

New nanogel comprising one or more bioactive molecule, therapeutic molecule or drug together with an amphiphilic molecule and method of preparation thereof. Biomaterial implant, medical device or bioprosthesis coated with the nanogel and method of producing thereof.

### FIELD OF THE INVENTION

The present invention relates to nanogel comprising bioactive molecules, therapeutic molecules or drugs together with an amphiphilic molecule, particularly vitamin E derivative. The present invention also relates to a medical device, biomaterial implant or bioprosthesis coated with the nanogel, a method of making such nanogel and a method of coating the medical device, biomaterial implant or bioprosthesis, particularly a catheter.

Most medical devices, biomaterial implants or bioprosthesis raise biocompatibility issues. Importantly, implantation of foreign materials in blood vasculature activates the contact pathway of coagulation, which may lead to thrombotic complications. For example, surface roughness of a medical device, biomaterial implants or bioprosthesis is an important factor influencing thrombogenicity.

Medical devices, biomaterial implants or bioprosthesis may also become infected and treatment for such infections generally requires administration of antibiotics targeting the causative bacteria.

Moreover, the increasing resistance to antibiotics has increased the demand for antibiotics exhibiting both antibacterial efficacy and anti-antibiotic resistance. As this demand has not been satisfied by a conventional one-target-one molecule approach, other approaches are required as the multi-target antibiotics.

Nanogels are known in the art. WO2018/122318A1 describes a nanogel made of a first hydrophilic polymer or copolymer bearing catechol groups and crosslinked with a second hydrophilic polymer bearing one or more reactive moieties; the nanogels also comprise bioactive molecules, therapeutic molecules or drugs.

Such nanogels are anchored or attached directly onto the surface of a medical device, biomaterial implant or bioprosthesis and are able to release the bioactive molecules, therapeutic molecules or drugs. Nevertheless, a difficulty in using such nanogels is the loading of water-insoluble or hydrophobic bioactive molecules, therapeutic molecules or drugs resulting in an irregular release kinetic profile and a lower pharmacological efficiency.

Moreover, only low levels of hydrophobic bioactive molecules, therapeutic molecules or drugs are released from such nanogel after release initiation. Immediate pharmacological effect can therefore not be rapidly achieved, and bacterial adhesion to the medical device, biomaterial implant or bioprosthesis cannot be fully prevented.

### SUMMARY OF THE INVENTION

We have now found an improved nanogel comprising one or more bioactive molecules, therapeutic molecules or drugs together with an amphiphilic molecule preferably a vitamin-E derivative or most preferably an amphiphilic cyclodextrin; the nanogel exhibiting improved antibacterial efficacy and anti-antibiotic resistance.

The present invention surprisingly and advantageously provides more efficient loading of hydrophobic bioactive molecules, therapeutic molecules or drugs in nanogel and a steady, progressive and continuous release over time during weeks. The hydrophobic bioactive molecules, therapeutic molecules or drugs can be combined with a hydrophilic bioactive molecules, therapeutic molecules or drugs in such nanogels, and be released simultaneously. Moreover, the nanogel comprising the bioactive molecules, therapeutic molecules or drugs advantageously remains stable over time in liquid suspension by maintaining its structural integrity and uniform distribution in dispersed state, particularly in aqueous media.

The nanogel of the invention is therefore for use in treatment or prevention of bacterial infection, particularly in topical administration for host mammal.

The use of such new nanogel in coating medical device, biomaterial implants or bioprosthesis, particularly catheter; also provides improved medical device, biomaterial implants or bioprosthesis.

The medical device, biomaterial implants or bioprosthesis coated with the nanogel of the invention, advantageously provides a more homogeneous, hydrophilic and smooth surface and content uniformity, resulting in a more reproducible kinetic release of the hydrophobic bioactive molecules, therapeutic molecules or drugs from the coated medical device, biomaterial implants or bioprosthesis, and subsequent improved pharmacological efficiency.

Simultaneously, medical device, biomaterial implants or bioprosthesis coated with the nanogel of the invention, advantageously reduces bacterial adhesion to the surface of the medical device, biomaterial implants or bioprosthesis, particularly when the nanogel comprises a combination of a bacteriostatic agent and an antivirulence or a bactericidal agent.

### DETAILED DESCRIPTION

The present invention relates to a new nanogel, a three-dimensional cross-linked particle with submicron particle size, which provide fora larger cargo space which may be used to incorporate bioactive molecule, therapeutic molecule or drug encapsulated by amphiphilic molecule, particularly Vitamin E derivatives or amphiphilic cyclodextrin.

Such nanogel for use in treatment and prevention of bacterial infection, particularly bacterial virulence, is particularly useful for topical administration on host mammal in need of such treatment

Such nanogel can also be anchored or attached onto the surface of any biomaterial or medical device, be it metallic or polymeric, or on a bioprosthesis and thereby reduce or prevent infection and improve biocompatibility and hemocompatibility of transiently or permanently implanted materials to help maintain their functionality and increase their durability.

According to a first aspect, the present invention provides a nanogel made of a poly(methacrylamide)-bearing quinone groups of formula (1)

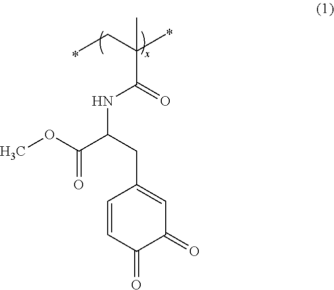

wherein x is an integer >1, preferably x is between 1 and 100;
said poly(methacrylamide) been crosslinked with a polymer bearing primary or secondary amine groups; and
wherein the nanogel comprises one or more bioactive molecule, therapeutic molecule or drug together with an amphiphilic molecule. The amphiphilic molecule is preferably a vitamin-E derivative, more preferably D-α-tocophenyl polyethylene glycol succinate. The amphiphilic molecule may also be an amphiphilic cyclodextrin preferably hydroxypropyl-beta-cyclodextrin.

Alternatively, the present invention also provides a nanogel made of a poly(vinylquinone) represented by formula (6)

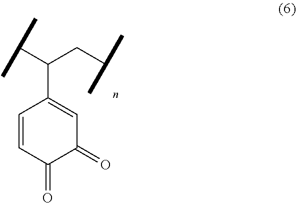

wherein n is an integer >1; preferably n is between 1 and 100
said poly(vinylquinone) been crosslinked with a polymer bearing primary or secondary amine groups; and
wherein the nanogel comprises one or more bioactive molecule, therapeutic molecule or drug together with an amphiphilic molecule. The amphiphilic molecule is preferably a vitamin-E derivative, more preferably D-α-tocophenyl polyethylene glycol succinate. The amphiphilic molecule may also be an amphiphilic cyclodextrin, preferably hydroxypropyl-beta-cyclodextrin.

Alternatively, the present invention also provides a nanogel made of a combination of both quinone polymers, or copolymer made of poly(methacrylamide) and poly(vinylquinone). The present invention also extends to all polymers or copolymers bearing quinone groups.

The poly(methacrylamide)-bearing quinone groups of formula (1) is obtained by oxidation of catechol group (also known as benzene 1,2 diol group) of P(mDOPA) of formula (2). The oxidation is preferably carried out in aqueous media under basic conditions at pH above 10, preferably at a pH between 10 and 12.

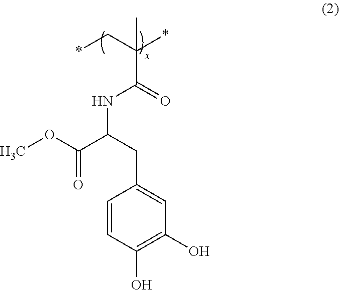

The poly(vinylquinone) of formula (6) is similarly obtained by oxidation of catechol group (also known as benzene 1,2 diol group) of poly(vinylcatechol) of formula (7)

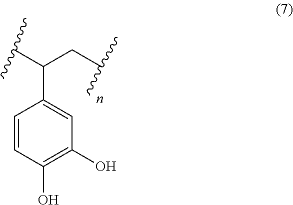

wherein n is an integer >1, preferably n is between 1 and 100.

The oxidation is preferably carried out in aqueous media under basic conditions, at pH above 10, preferably at a pH between 10 and 12.

The amphiphilic molecule is a molecule possessing both hydrophilic and lipophilic properties. Amphiphilic molecules have the property of self-assembly for example into micelles when dispersed in water. They may be a surfactant such as sodium dodecylsulfate, 1-octanol, cocamidopropyl betaine, benzalkonium chloride, phospholipids, cholesterol, glycolipids, fatty acids, a vitamin-polyether copolymer such as vitamin E-polyoxyalkylene copolymer, particularly D-α-tocopheryl polyethylene glycol succinate (TPGS). The amphiphilic molecule also refers to amphiphilic cyclodextrin obtained by grafting hydrocarbonated chains in the hydroxyl groups of cyclodextrin such as for example hydroxypropyl beta-cyclodextrin or sulfobutylether beta-cyclodextrin. The amphiphilic cyclodextrin is able to incorporate an hydrophobic biological molecule, therapeutic molecule or drug into its hydrophobic cavity.

For sake of clarity, the amphiphilic molecule does not extend to a molecule bearing organic moiety as well as hydrophilic group and belonging to bioactive molecule, therapeutic molecule or drugs as defined in the present invention. For example vancomycine, minocycline or ticagrelor are not considered as amphiphilic molecules.

The polymer bearing primary or secondary amine groups may be a polyallylamine, a polyvinylamine, a polyvinylamide, a polyvinylalcohol, a poly(meth)acrylate, a poly(meth)acrylamide, a polyurethane, a polyethylene glycol (PEG), a polyelectrolyte (cationic, anionic or zwitterionic) with reactive groups that are primary or secondary amines;

The polymer bearing primary or secondary amine groups may be a natural or a synthetic polymer with a primary or secondary amine function, for example polyvinyl amine, chitosan or a protein.

In a preferred embodiment, the polymer bearing primary amine groups may comprise a polyallylamine, such as poly-(allylamine hydrochloride) also known as PAH, as illustrated below wherein p is an integer >1, preferably p is between 10 and 300:

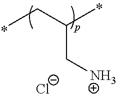

The bioactive molecule, therapeutic molecule or drug may be antibiotics, anti-biofilm formation agents, anti-platelet agents, anti-coagulants, anti-thrombotic agents, and anti-calcification agents.

Bioactive agents (also called bioactive molecules) are molecules derived from plant, seeds, fungi, animals, human or microorganisms or can be synthetically produced. They may include any agent which is desired to be delivered to molecules, cells, tissues or organs for modulating or otherwise modifying molecule or cell function, including for therapeutic effects. Bioactive agents include, but are not limited to, pharmaceutically active compounds or diagnostic compounds. Bioactive molecules or bioactive compounds include, but are not limited to, nucleotides (aptamers, RNAi, antisense oligonucleotides), peptides, oligopeptides, proteins, apoproteins, glycoproteins, antigens and antibodies or antibody fragments thereto, receptors and other membrane proteins, retro-inverso oligopeptides, protein analogs in which at least one non-peptide linkage replaces a peptide linkage, enzymes, coenzymes, enzyme inhibitors, amino acids and their derivatives, hormones, lipids, phospholipids, liposomes, ricin or ricin fragments; toxins such as aflatoxin, digoxin, xanthotoxin, rubratoxin; analgesics such as aspirin, ibuprofen and acetaminophen; bronchodilators such as theophylline and albuterol; beta-blockers such as propranolol, metoprolol, atenolol, labetolol, timolol, penbutolol and pindolol; antimicrobial agents such as those described above and ciprofloxacin, cinoxacin and norfloxacin; antihypertensive agents such as clonidine, methyldopa, prazosin, verapamil, nifedipine, aptopril and enalapril; cardiovascular agents including antiarrhythmics, cardiac glycosides, antianginals and vasodilators; central nervous system agents including stimulants, psychotropics, antimanics and depressants; antiviral agents; antihistamines such as chlorphenirmine and brompheniramine; cancer drugs including chemotherapeutic agents, such as chlorambucil, carboplatin, derivatives of busulfan, doxorubicin, etoposide, topotecan (TPT); tranquilizers such as diazepam, chordiazepoxide, oxazepam, alprazolam and triazolam, anti-depressants such as fluoxetine, amitriptyline, nortriptyline and imipramine; H-2 antagonists such as nizatidine, cimetidine, famotidine and ranitidine; anticonvulsants; antinauseants; prostaglandins; muscle relaxants; anti-inflammatory substances; stimulants; decongestants; antiemetics; diuretics; antispasmodics; antiasthmatics; anti-Parkinson agents; expectorants; cough suppressants; mucolytics; vitamins; and mineral and nutritional additives. Other molecules include nucleotides; oligonucleotides; polynucleotides; and their art-recognized and biologically functional analogs and derivatives including, for example, methylated polynucleotides and nucleotide analogs having phosphorothioate linkages; plasmids, cosmids, artificial chromosomes, other nucleic acid vectors; antisense polynucleotides including those substantially complementary to at least one endogenous nucleic acid or those having sequences with a sense opposed to at least portions of selected viral or retroviral genomes; promoters; enhancers; inhibitors; other ligands for regulating gene transcription and translation.

The bioactive agent may be an anti-infective agent. Anti-infective agents include, but are not limited to antibiotics, such as amikacin, gentamicin, kanamycin, neomycin, netilmicin, tobramycin, paromomycin, streptomycin, spectinomycin, geldanamycin, herbimycin, rifaximin, loracarbef, ertapenem, dorpenem, imipenem/cilastatin, meropenem, cefadroxil, cefazolin, cefalotin, cephalexin, cefaclor, cefamandole, cefoxitin, cefproxil, cefuroxime, cefixime, cefdinir, cedfitoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, daibavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, amoxicillin, ampicillin, piperacillin, ticarcillin, bacitracin, colistin, polymyxin B, ciprofloxacin, enoxacin, gatifloxacin, gemifloxacin, levoflaxicin, lomefloxacilin, moxifloxacin, nalidixic acid, norfloxacin, ofloxacin, mafenide, sulfacetamide, sulfadizine, silver sulfadizine, sulfadimethoxine, sulfamethizole, sulfamethoxazole, sulfanilimide, sulfisoxazole, trimethoprim-sulfamethoxazole, sulfonamidochrysoidine, demeclocyline, doxycycline, minocycline, oxytetracycline, tetracycline, clofazimine, dapsone, rifampicin, rifabutin, arspehnamine, chloramphenicol, fosfomycin, metronidazole, thiamphenicol, tigecycline, tinidazole, and trimethoprim.

Antibiotics may be either bacteriostatic to inhibit growth without killing or bactericidal for killing. The following antibiotics of the above-mentioned list are generally considered as bacteriostatic: minocycline (particularly for S. aureus), tetracyclines, macrolides, clindamycin, linezolid, chloramphenicol, chlorhexidine or alexidine when used at low level or a combination thereof, whereas the other antibiotics of the list above are most often bactericidal.

Antibiotics may also be antivirulence agents, when provided at low level.

Virulence factors are molecules produced by pathogens that allow colonization, immunoevasion, damaging host cells. Antivirulence agent targets virulence factors of pathogens instead of killing or stopping their growth, and consequently disarm infectious pathogens. In contrast to bactericidal antibiotics that drives resistance, antivirulence agents do not create a selective pressure driving resistance. Antivirulence agents interfere in interaction of the pathogen, particularly bacteria with host mammal, and thereby reduces damage to the host and impair bacteria ability to cause disease. Antivirulence agents can inhibit bacterial toxin production or prevent their adhesion to tissues.

Anti-biofilm formation agents include, but are not limited to naturally occurring peptides such as human cathelicidin LL-37 or the bovine peptide indolicidin, or synthetic peptides such as 1018, natural compounds with 2-aminoimidazole moiety, 2-aminoimidazole based inhibitors, benzimidazoles analogs, indole-triazo-amide analogs, plant-derived biofilm inhibitors such as emodin, phloretin, casbane diterpene, resveratrol and its oligomers, sulphur derivatives, brominated furanone analogs, bromopyrrole alkaloids, skyllamycins and (−)-ageloxime D structures, cembranoids, N-acyl homoserine lactone analogs, carolacton, molecules that interfere with the formation of amyloid-like fibres, fatty acids, nitric oxide donors, ionic liquids as 1-alkyl-3-methyl imidazolium chloride, 1-alkylquinolinium bromide, all these agents can be used in combination with conventional antibiotics.

Anti-platelet agents include, but are not limited to, irreversible cyclooxygenase inhibitors such as aspirin and, triflusal (Disgren), adenosine diphosphate (ADP) receptor inhibitors such as clopidogrel (Plavix), prasugrel (Effient), ticagrelor (Brilique and Brilinta), ticlopidine (Ticlid), Phosphodiesterase inhibitors such as cilostazol (Pletal), Protease-activated receptor-1 (PAR-1) antagonists such as vorapaxar (Zontivity), glycoprotein llB/lIlA inhibitors (intravenous use only) such as abciximab (ReoPro), eptifibatide (Integrilin), tirofiban (Aggrastat), Adenosine reuptake inhibitors such as dipyridamole (Persantine), thromboxane inhibitors, thromboxane synthase inhibitors and thromboxane receptor antagonists such as terutroban, glycoprotein VI inhibitors such as Revacept, glycoprotein Ib inhibitors, and von Willebrand factor inhibitors.

Anti-coagulants include, but are not limited, to acenocoumarol, coumatetralyl, dicoumarol, ethyl biscoumacetate, phenprocoumon, warfarin, clorindione, dipjenadione, phenindione, ticlomarol, bemiparin, certoparin, ardeparin, dalteparin, enoxaparin, nadroparin, parnaparin, reviparin, dabigatran, apixaban, betrixabaan, darexaban, edoxaban, otamixaban, rivaroxaban, alteplase, danaparoid, tinzaparin, and fondaparinux.

Thrombolytic agents include, but are not limited to, alteplase, reteplase, tenecteplase, saruplase, urokinase, anistreplase, monteplase, streptokinase, ancrod, brinase and fibrinolysin.

Anti-calcification agents include, but are not limited to, bisphosphonates, aluminium salts, glutaraldehyde, amino oleic acid, and metalloproteinase inhibitors.

In a preferred embodiment, the nanogel according to the invention comprises an antibiotic and/or an antiplatelet agent.

In another preferred embodiment, the nanogel according to the invention comprises a combination of antibacterial agents, more preferably a bacteriostatic agent with an antivirulent agent inhibiting bacterial growth and bacterial adhesion when the nanogel is coated on a surface.

Advantageously, the nanogel comprising such combination of a bacteriostatic and antivirulence agents delays bacterial growth, as measured by metabolic rate and increase anti-adhesive property when the nanogel is coated on a medical device, a biomaterial implant or bioprosthesis. Preferably the molecular ratio of the bacteriostatic agent to the antivirulene agent is between 1:1 to 1:10, more preferably 1:2

In a more preferred embodiment, the bacteriostatic agent is minocycline.

In another preferred embodiment, the bacteriostatic agent is chlorhexidine.

In a preferred embodiment, the bioactive molecule, therapeutic molecule or drug is a triazolo(4,4-d)-pyrimidine derivative of formula (3)

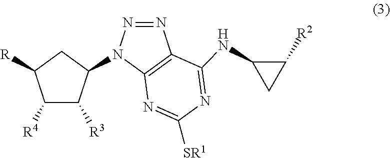

wherein R1 is C3-5 alkyl optionally substituted by one or more halogen atoms; R₂ is a phenyl group, optionally substituted by one or more halogen atoms; R₃ and R₄ are both hydroxyl; R is OH or XOH, wherein X is CH₂, OCH₂CH₂, or a bond;
or a pharmaceutical acceptable salt or solvate thereof, or a solvate thereof or a solvate of such a salt provided that when X is CH₂ or a bond, R₁ is not propyl; when X is CH₂ and R₁ CH₂CH₂CF₃, butyl or pentyl, the phenyl group at R₂ must be substituted by fluorine; when X is OCH₂CH₂ and R₁ is propyl, the phenyl group at R₂ must be substituted by fluorine.

The triazolo(4,4-d)-pyrimidine derivatives of formula (3) have advantageously an antiplatelet, but also an antibacterial effect. It is particularly useful to reduce or prevent infection of blood-contacting medical device, biomaterial implants or bioprosthesis, when inserted or implanted into a mammal host, and to prevent thrombosis. Thrombosis indeed promotes infection of blood-contacting medical device, biomaterial implants or bioprosthesis. The mammal host may be a human patient or an animal.

In a most preferred embodiment the triazolo (4,4-d)-pyrimidine derivative is (1S,2S,3R,5S)-3-[7-[(1R,2S)-2-(3,4-difluorophenyl)cyclopropylamino]-5-(propylthio)-3H-[1,2,3]-triazolo[4,5-d]pyrimidin-3-yl]-5-(2-hydroxyethoxy)-1,2-cyclopentanediol) also called Triafluocyl.

In another most preferred embodiment the triazolo (4,4-d)-pyrimidine derivative is (1S,2R,3S,4R)-4-[7-[[(1R,2S)-2-(3,4-Difluorophenyl)-cyclopropyl]amino]-5-(propylthio)-3H-1,2,3-triazolo[4,5-d]pyrimidin-3-yl]-1,2,3-cyclopentanetriol also called Fluometacyl or Fluometacyl® and illustrated in formula (5).

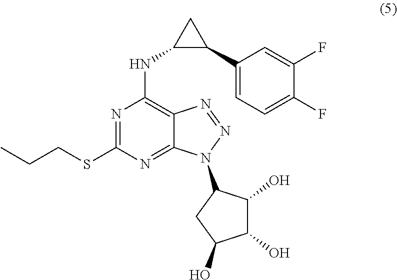

In another preferred embodiment, the bioactive molecule, therapeutic molecule or drug is a pyrimidine derivative represented by formula (4)

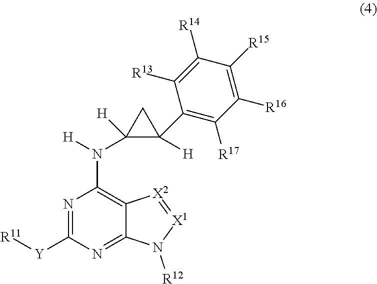

or optical isomers, racemic mixtures thereof, pharmaceutically acceptable acid addition salts, pharmaceutically acceptable metal salts, or alkylated ammonium salts or prodrug thereof;
wherein:
X¹ and X² are independently N, CH, CR⁸ wherein R⁸ is C₁₋₆ alkyl, C₂₋₆ alkenyl or C₂₋₆ alkynyl; with the exception that if one of X¹ or X² is equal to N, then the remaining X¹ or X² are selected from CH, CR⁸.
—Y— is —O— or —S—;
R¹¹ and R¹² are independently C₁₋₆-alkyl, C₂₋₆-alkenyl, C₂₋₆-alkynyl, C₃₋₆-cycloalkyl, aryl, aryl-C₁₋₆-alkyl wherein the alkyl or cycloalkyl moiety is optionally mono or polysubstituted with OH or an halogen and the aryl moiety is optionally mono or polysubstituted with an halogen, —C₁₋₆ alkyl, —C₁₋₆ alkoxy, —OH, —NO₂, —CN, —NH₂, —NHR⁸, —N(R⁸)₂—COOH, —COOR⁸, —CONH₂, —CONHR⁸, —CON(R⁸)₂, —SO₂NH₂, —SO₂NHR⁸, or —SO₂N(R⁸)₂;
R¹³, R¹⁴, R¹⁵, R¹⁶ and R¹⁷ are independently H, an halogen, a C₁₋₆alkyl, C₁₋₆alkoxy, —OH, —NO₂, —CN, —NH₂, —NHR⁸, —N(R⁸)₂—COOH, —COOR⁸, —CONH₂, —CONHR⁸, —CON(R⁸)₂, —SO₂NH₂, —SO₂NHR⁸, or —SO₂N(R⁸)₂.

The pyrimidine derivatives of formula (4) have advantageously an anti-bacterial effect. They are particularly useful to reduce or prevent infection of medical device, biomaterial implants or bioprosthesis when inserted or implanted into a mammal host. The mammal host may be a human patient or an animal.

In another preferred embodiment, the bioactive molecule, therapeutic molecule or drug is a bisguanide, preferably chlorhexidine also called hereafter chlorhexidine chloride.

Biguanide or HN(C(NH)NH₂)₂ as used herein refers to formula (7)

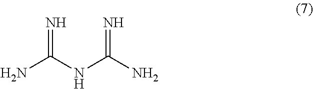

Bisbiguanide may be chlorhexidine, alexidine, and polyhexylbiguanide;
Chlorhexidine as used herein refers to chlorhexidine base (8) also called chlorhexidine chloride.

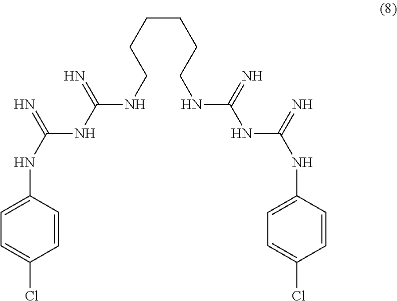 [SMILES: N=C(NCCCCCCNC(=N)NC(=N)Nc1ccc(Cl)cc1)NC(=N)Nc1ccc(Cl)cc1]

but can also refer to a chlorhexidine salt such as for example chlorhexidine di phosphanilate, chlorhexidine digluconate, chlorhexidine diacetate, chlorhexidine dinitrate, chlorhexidine dihydrochloride, chlorhexidine dichloride, chlorhexidine acetate, chlorhexidine dipropionate, chlorhexidine maleate, chlorhexidine succinate, chlorhexidine thiosulfate, chlorhexidine di-acid phosphate, chlorhexidine malate, chlorhexidine dibenzoate, chlorhexidine diisophtalate, chlorhexidine dilaurate, chlorhexidine distearate, and the like.

Alexidine as used herein refers to alexidine base,
but may also refer to alexidine hydrochloride, alexidine dihydrochloride, alexidine monoacetate, alexidine diacetate, alexidine gluconate, alexidine digluconate and mixture thereof.

The bioactive agent, therapeutic molecule or drug is dispersed in a solvent together with an amphiphilic molecule, preferably a vitamin-E derivative or an amphiphilic cyclodextrin, more preferably hydroxypropyl-beta-cyclodextrin, before addition into the resulting dispersion of poly(methacrylamide)-bearing quinone groups or poly(vinylquinone).

The solvent may be any solvent comprising O—H group. The solvent should not contain N—H and/or —SH bond to avoid an interaction with a catechol group. The solvent is for example water, alcohol such as methanol, ethanol, butanol, propanol and the like, or a combination thereof.

The vitamin-E derivative may be any copolymer obtained by esterification of Vitamin-E (also called α-tocopherol succinate) with an acid ester such as Vitamin E acetate or with a linear or branched polyether such as polyoxyalkylene as for example, polyoxyethylene, polyoxypropylene, polyoxypropylene-polyoxyethylene copolymer, polyethylene glycol, polypropylene glycol, and the like.

The polyalkylene glycol have a molecular weight between 500 and 2000, preferably between 750 and 1000, most preferably 1000.

In a preferred embodiment Vitamin-E derivative is
D-α-tocopheryl polyethylene glycol succinate (TPGS), represented in formula A.

TPGS is a copolymer obtained by esterification with polyethylene glycol (PEG 750 or PEG1000) of Vitamin-E (also called α-tocopherol succinate)

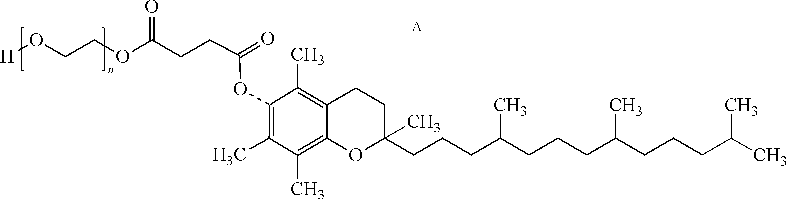

The amphiphilic molecule, particularly vitamin E derivatives, more particularly TPGS copolymer forms efficiently micelles into solvent such as water or an aqueous solution containing 0 to 60% of alcohol as for example ethanol. TPGS encapsulates hydrophobic bioactive agent, therapeutic molecule or drug and increase the loading of such hydrophobic bioactive agent, therapeutic molecule or drug into the nanogel, but also their efficiency and bioavailability by a continuous release from within the nanogel over weeks, preferably more than 10 days.

TPGS copolymer forms efficiently micelles with hydrophobic bioactive molecule, therapeutic molecule or drug into an aqueous solution; when mixed in a ratio TPGS: (bioactive agent, therapeutic molecule or drug) of 1:1 w/w to 5:1 w/w; preferably 2:1 w/w.

The hydrophilic moiety of TPGS copolymer, polyethylene glycol (PEG), forms the corona of the micelles whereas the hydrophobic moiety, tocopherol succinate forms their core. The hydrophobic core of micelles can solubilize poorly soluble or insoluble drugs and partly protect the bioactive agent, therapeutic molecule or drug from aqueous environment. TPGS molecules encapsulate the hydrophobic bioactive agent, therapeutic molecule or drug and contribute to their better stability when inserted into the nanogel.

The micelles obtained by such encapsulation of the bioactive agent, therapeutic molecule or drug have a mean particles size in the range of 10 to 100 nm, preferably 10 nm.

The bioactive agent, therapeutic molecule or drug together with an amphiphilic molecule, preferably a TPGS, is encapsulated in micelles and is entrapped in the nanogel.

The nanogel comprises micelles of amphiphilic molecule preferably TPGS, together with bioactive agent, therapeutic molecule or drug. The diameter of the nanogel is less than 1000 nm, e.g. about 100 nm to 300 nm. For example, the nanogel may have a diameter of less than about 500 nm, less than about 300 nm, less than about 200 nm, less than about 150 nm. In particular embodiments, the nanogel of the present invention has a diameter of about 150 nm to about 250 nm. In particular embodiments, the nanogel of the present invention has a diameter of about 100 to about 250 nm.

The nanogel may also comprise an amphiphilic molecule as amphiphilic cyclodextrin (Cy) structure or vesicle incorporating hydrophobic bioactive agent, therapeutic molecule or drug into the Cy hydrophobic cavity.

When the micelles or vesicles are loaded into the nanogel, the amphiphilic molecule, preferably TGPS, more preferably hydroxy-propyl-beta-cyclodextrin surprisingly reduce bacterial adhesion to coated medical device, biomaterial implants or bioprosthesis and does not prevent the pharmacological effect of the bioactive agent, therapeutic molecule or drug as it can in a solvent mixture.

The nanogel according to the invention can advantageously load a higher level of bioactive molecule, therapeutic molecule or drug. Consequently, the nanogel according to the invention also advantageously allows a more prolonged release in contact with cells, tissues or organs when coated on medical device, biomaterial implants or bioprosthesis.

The nanogel according to the invention can advantageously load hydrophilic and hydrophobic bioactive agent, therapeutic molecule or drug together. Preferably the hydrophilic to hydrophobic bioactive agents, therapeutic molecules or drugs are in a molecular ratio from 1:0 to 1:1; preferably 1:0.5

The nanogel according to the invention maintain its structural integrity and prevent aggregation or degradation over time. Stable nanogels maintain their dispersed state, ensuring uniform distribution and optimal performance. A stable nanogel structure enhances the controlled release of drugs,

According to a second aspect, the present invention provides methods of making the nanogel comprising one or more bioactive molecules, therapeutic molecules or drugs, together with an amphiphilic molecule, wherein the nanogel is obtained by one of both following methods, depending whether each bioactive molecule, therapeutic molecule or drug is loaded separately or simultaneously with the amphiphilic molecule in the nanogel:
a) when each bioactive molecule, therapeutic molecule or drug is loaded separately with the amphiphilic molecule, the method comprises the following in-sequence steps:
   i) mixing poly(methacrylamide)-bearing quinone groups of formula (1)

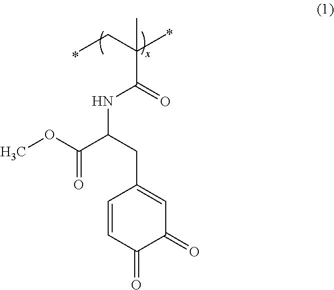

wherein x is an integer >1, preferably x is between 1 and 100 with only one bioactive molecule, therapeutic molecule or drug together with an amphiphilic molecule, preferably vitamin-E derivative or an amphiphilic cyclodextrin;
   ii) adding a solution of polymer bearing primary or secondary amine groups to the resulting mixture obtained in step i) to generate a crosslinked nanogel comprising one bioactive molecule, therapeutic molecule or drug together with an amphiphilic molecule, preferably a vitamin-E derivative or an amphiphilic cyclodextrin;
   iii) repeating steps 1) and ii) for each additional bioactive molecule, therapeutic molecule or drug;
   iv) mixing each crosslinked nanogel obtained at step iii) to obtain a resulting nanogel comprising one or more biological molecule, therapeutic molecule or drug.
b) when the bioactive molecules, therapeutic molecules or drugs are loaded simultaneously with the amphiphilic molecule, the method comprises the following in-sequence steps:
   i) mixing poly(methacrylamide)-bearing quinone groups of formula (1)

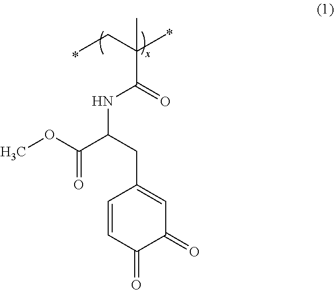

wherein x is an integer >1, preferably x is between 1 and 100
with one or more bioactive molecules, therapeutic molecules or drugs together with an amphiphilic molecule, preferably vitamin-E derivative or an amphiphilic cyclodextrin;
   ii) adding a solution of polymer bearing primary or secondary amine groups to the resulting mixture obtained in step i) to generate crosslinked-nanogel comprising one or more bioactive molecule, therapeutic molecule or drug together with an amphiphilic molecule, preferably a vitamin-E derivative or an amphiphilic cyclodextrin;

In the first step (i), micelle or vesicle formation occurs directly after addition of a solution of poly(methacrylamide)-bearing quinone groups of formula (1) to the mixture of one or more bioactive molecules, therapeutic molecules or drugs together with an amphiphilic molecule, preferably a vitamin-E derivative or an amphiphilic cyclodextrin in a solvent. The solvent may be water or alcohol or a combination thereof. The solvent is preferably ethanol. The solution of poly(methacrylamide)-bearing quinone groups may be water or alcohol or a combination thereof, but is preferably water.

The addition is carried out under stirring at room temperature.

In the second step (ii), the polymer bearing primary or secondary amine groups reacts through a quinone-amine reaction, with poly(methacrylamide)-bearing quinone groups and of formula (1) to generate a nanogel comprising one or more bioactive molecules, therapeutic molecules or drugs with an amphiphilic molecule, preferably a vitamin-E derivative or an amphiphilic cyclodextrin in the solvent.

Alternatively, the present invention provides a method of making the nanogel comprising bioactive molecules, therapeutic molecules or drugs, together with an amphiphilic molecule, wherein the nanogel is obtained by one of both following methods, depending whether each bioactive molecule, therapeutic molecule or drugs is loaded separately or simultaneously with the amphiphilic molecule in the nanogel:
a) when each bioactive molecule, therapeutic molecule or drugs is loaded separately with the amphiphilic molecule, the method comprises the following in-sequence steps of:
   i) mixing poly(vinyl) quinone groups of formula (6)
with only one bioactive molecule, therapeutic molecule or drug together with an amphiphilic molecule, preferably vitamin-E derivative or an amphiphilic cyclodextrin;
   ii) adding a solution of polymer bearing primary or secondary amine groups to the resulting mixture obtained in step i) to generate a crosslinked nanogel comprising one bioactive molecule, therapeutic molecule or drug together with an amphiphilic molecule, preferably a vitamin-E derivative or an amphiphilic cyclodextrin;
   iii) repeating step i) and ii) for each additional bioactive molecule, therapeutic molecule or drug;
   iv) mixing each crosslinked nanogel obtained at step iii) to obtain a resulting nanogel comprising two or more biological molecule, therapeutic molecule or drug.
b) when the bioactive molecules, therapeutic molecules or drugs are loaded together with the amphiphilic molecule, the method comprises the following in-sequence steps:
   i) mixing a poly(vinyl)quinone of formula (6)

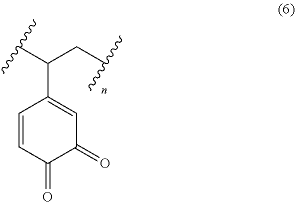

wherein n is an integer >1 preferably n is between 1 and 100;
with one or more bioactive molecule, therapeutic molecule or drug together with an amphiphilic molecule, preferably a vitamin-E derivative or an amphiphilic cyclodextrin;
   ii) adding a solution of polymer bearing primary or secondary amine groups to the resulting mixture obtained in step i) to generate a crosslinked nanogel comprising one or more bioactive molecule, therapeutic molecule or drug together with an amphiphilic molecule, preferably vitamin-E derivative or an amphiphilic cyclodextrin.

In the first step (i), micelle or vesicle formation and encapsulation with the amphiphilic molecule occurs directly after addition of a solution of poly(vinyl)quinone of formula (6) to the mixture of one or more bioactive molecules, therapeutic molecules or drugs with an amphiphilic molecule, preferably a vitamin-E derivative or an amphiphilic cyclodextrin in a solvent.

The solvent may be water or alcohol or a combination thereof, but is preferably ethanol.

The solution of poly(vinylquinone) may be water or alcohol or a combination thereof, but is preferably water.

The addition is carried out under stirring at room temperature.

In the second step (ii), the polymer bearing primary or secondary amine groups react through a quinone-amine interaction, to the poly(vinylquinone) of formula (6) to generate nanogel comprising one or more bioactive molecule, therapeutic molecule or drug encapsulated with the amphiphilic molecule, preferably the vitamin-E derivative or an amphiphilic cyclodextrin.

In a preferred embodiment, the vitamin-E derivative is D-α-tocophenyl polyethylene glycol succinate.

In another preferred embodiment, the amphiphilic cyclodextrin is hydroxypropyl beta-cyclodextrin.

In another preferred embodiment, the polymer bearing primary or secondary amine groups comprises a polyallylamine, preferably poly-(allylamine hydrochloride) also known as PAH, as illustrated below wherein p is an integer >1, preferably p is between 10 and 300, most preferably p is 160:

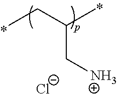

The solvent may be any solvent comprising a 0-H group and therefore at least one hydrogen susceptible to interact in Hydrogen bonding. They are for example water, alcohol such as methanol, ethanol, butanol, propanol and the like, or a combination thereof.

In a preferred embodiment the solvent is an alcohol, preferably ethanol.

In a preferred embodiment the bioactive molecule, therapeutic molecule or drug is a triazolo(4,4-d)-pyrimidine derivative of formula (3), most preferably the triazolo(4,4-d)-pyrimidine derivative is (1S,2S,3R,5S)-3-[7-[(1R,2S)-2-(3,4-difluorophenyl)cyclopropylamino]-5-(propylthio)-3H-[1,2,3]-triazolo[4,5-d]pyrimidin-3-yl]-5-(2-hydroxyethoxy)-1,2-cyclopentanediol) also called triafluocyl or (1S,2R,3S,4R)-4-[7-[[(1R,2S)-2-(3,4-Difluorophenyl)-cyclopropyl]amino]-5-(propylthio)-3H-1,2,3-triazolo[4,5-d]pyrimidin-3-yl]-1,2,3-cyclopentanetriol also called Fluometacyl or Fluometacyl®.

In another preferred embodiment, the bioactive molecule, therapeutic molecule or drug is a bisguanide, most preferably chlorhexidine.

In still another preferred embodiment the triazolo(4,4-d)-pyrimidine derivative, is combined with a bacteriostatic antibiotic, preferably minocycline. The triazolo(4,4-d)-pyrimidine derivative is preferably Triafluocyl or Fluometacyl or Fluometacyl®.

In still another preferred embodiment the triazolo(4,4-d)-pyrimidine derivative, is combined with a bisguanide, most preferably chlorhexidine According to a third aspect, the present invention provides a biomaterial implant, medical device or bioprosthesis wherein a surface or part thereof is coated with the nanogel made of a poly-(methacrylamide)-bearing quinone groups of formula (1)

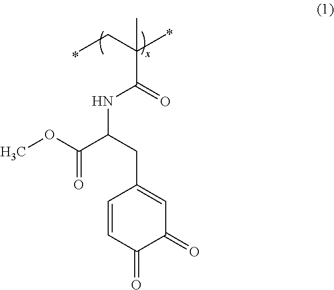

wherein x is an integer >1, preferably in the range between 1 and 100
said poly-(methacrylamide) been crosslinked with a polymer bearing primary or secondary amine groups; and
wherein the nanogel comprises one or more bioactive molecule, therapeutic molecule or drug together with an amphiphilic molecule, preferably vitamin-E derivative, more preferably D-α-tocophenyl polyethylene glycol succinate.

Alternatively, the present invention provides a biomaterial implant, medical device or bioprosthesis wherein a surface or part thereof is coated with the nanogel made of a poly(vinyl)quinone of formula (6)

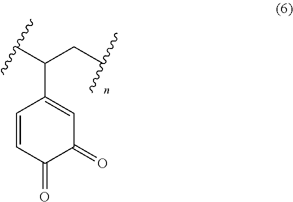

wherein n is an integer >1, preferably in the range between 1 and 100; said poly(vinyl)quinone been crosslinked with a polymer bearing primary or secondary amine groups; and
wherein the nanogel comprises one or more bioactive molecule, therapeutic molecule or drug together with an amphiphilic molecule, preferably a vitamin-E derivative, more preferably D-α-tocophenyl polyethylene glycol succinate.

Alternatively, the present invention provides a biomaterial implant, medical device or bioprosthesis wherein a surface or part thereof is coated with the nanogel made of poly-(methacrylamide)-bearing quinone and polyvinylquinone or the copolymer thereof.

The present invention also extends to all biomaterial implant, medical device or bioprosthesis wherein a surface or part thereof is coated with the nanogel made of polymers or copolymers bearing quinone groups.

Biomaterial implant, medical device or bioprosthesis coated with the nanogel according to the invention advantageously provides a more homogeneous, hydrophilic and smooth surface

Such more homogeneous, hydrophilic and smooth surface of biomaterial implant, medical device or bioprosthesis coated with the nanogel advantageously reduces injuries, when inserted into human patient or animal and reduces thrombogenicity

Biomaterial implant, medical device or biosprosthesis coated with the nanogel comprising bioactive molecule, therapeutic molecule or drug together with amphiphilic molecule also advantageously provides a more reproducible kinetic release of the hydrophobic bioactive molecules, therapeutic molecules or drugs from the coated nanogel and from the medical device, biomaterial implants or bioprosthesis, resulting in a better pharmacological efficiency of bioactive molecules, therapeutic molecules or drugs, particularly to prevent infection and thrombotic complications.

Moreover, biomaterial implant, medical device or bioprosthesis coated with the nanogel according to the invention also advantageously provides reduced bacterial adhesion at their surface.

When the micelles comprising one or more bioactive molecule, therapeutic molecule or drug encapsulated with an amphiphilic molecule, preferably a vitamin-E derivative, more preferably D-α-tocophenyl polyethylene glycol succinate; are loaded into the nanogel coated on the surface of the Biomaterial implant, medical device or bioprosthesis; bacterial adhesion is reduced on the surface of the biomaterial implant, medical device or bioprosthesis. Such anti-adhesion effect is synergistically enhanced when a bacteriostatic agent is present into the nanogel

Moreover, the amphiphilic molecule, particularly TPGS in the nanogel coating does not prevent bioactive agent, therapeutic molecule or drug pharmacological effect as it may do when used in solution

A biomaterial implant may be any implantable foreign material for clinical use in host mammals such as for prosthetic joints, pacemakers, implantable cardioverter-defibrillators, catheters including intravascular or urinary catheters or materials, stents including coronary stents, mechanical and biological prosthetic heart valves, intraocular lens, dental implants and the like.

A medical device may be, but is not limited to, any device, tool, instrument, implant, or the like, relating to medical field or the practice of human or veterinary medicine, or intended for use to prevent or treat a disease. A medical device may include all natural and synthetic materials and both fibrous and non-fibrous materials. For example, the materials may be comprised of a metal, plastic, paper, glass, ceramic, textile, rubber, polymer, composite material or any other material or combination of materials. Exemplary medical devices include, but are not limited to, any kind of catheter; cannulae; needles; stents of any size, shape, or placement; coils of any size, shape, or placement; contact lenses; Intrauterine devices (IUDS); peristaltic pump chambers; endotracheal tubes; gastroenteric feeding tubes; arteriovenous shunts; condoms; oxygenator and kidney membranes; gloves; pacemaker leads; wound dressings; metallic pins, plates and screws; metallic artificial hips; artificial knees; and gels. In an embodiment, the nanogel of the invention may be used to coat a catheter to prevent bacterial infections.

A bioprosthesis may be, but is not limited to, a prosthesis made of biological material. Examples include heart valves, pericardium, vascular grafts, urinary bladder prostheses, tendon prostheses, hernia patches, surgical mesh and skin substitutes.

In an embodiment, the nanogel of the invention may be used to coat a bioprosthetic heart valve, for example a decellularized porcine heart valve or a bovine pericardium; to prevent bacterial infections and thrombosis.

The coated biomaterial implant, medical device or bioprosthesis may be used in human or animal host for diagnostic, to prevent or treat disease or for medical practice.

In a preferred embodiment the polymer bearing primary or secondary amine groups is poly-(allylamine hydrochloride) of formula (2) wherein p is an integer >1 preferably between 10 and 300.

In another preferred embodiment the bioactive molecule, therapeutic molecule or drug is a triazolo(4,4-d)-pyrimidine derivative of formula (3); preferably (1S,2S,3R,5S)-3-[7-[(1R,2S)-2-(3,4-difluorophenyl)cyclopropylamino]-5-(propylthio)-3H-[1,2,3]-triazolo[4,5-d]pyrimidin-3-yl]-5-(2-hydroxyethoxy)-1,2-cyclopentanediol) also called Triafluocyl or ticagrelor; or
is (1S,2R,3S,4R)-4-[7-[[(1R,2S)-2-(3,4-Difluorophenyl)-cyclopropyl]amino]-5-(propylthio)-3H-1,2,3-triazolo[4,5-d]pyrimidin-3-yl]-1,2,3-cyclopentanetriol also called Fluometacyl or Fluometacyl® that have both anti-bacterial and antiplatelet properties.

In another preferred embodiment the bioactive molecule, therapeutic molecule or drug is a bisguanide, more preferably chlorhexidine.

In another preferred embodiment the triazolo(4,4-d)-pyrimidine derivative is combined with a bacteriostatic antibiotic, preferably minocycline. Most preferably the triazolo(4,4-d)-pyrimidine derivative is Triafluocyl or Fluometacyl or Fluometacyl®.

In another preferred embodiment, the triazolo(4,4-d)-pyrimidine derivative is combined with a bisguanide, more preferably chlorhexidine.

The nanogel according to the invention may be anchored or attached onto the surface of the biomaterial implant, medical device or bioprosthesis using various physical or chemical methods known in the art. It is for example electrografting, layer-by-layer deposition, spin-coating, spraying or simply dipping the surface of biomaterial implant, medical device or bioprosthesis into a mixed solution of poly(methacrylamide)-bearing quinone groups of formula (1) with a mixture of bioactive molecule, therapeutic molecule or drug together with an amphiphilic molecule, preferably a vitamin-E derivative.

Alternatively dipping the surface of biomaterial implant, medical device or bioprosthesis into a mixed solution of poly(vinylquinone) with a mixture of one or more bioactive molecule, therapeutic molecule or drug together with an amphiphilic molecule, preferably a vitamin-E derivative is also possible.

The bioactive molecule, therapeutic molecule or drug is loaded into the nanogel in an encapsulated form with the amphiphilic molecule, preferably TPGS and is progressively and continuously release over time from the biomaterial implant, medical device or bioprosthesis into mammal host during weeks, preferably 2 weeks. The mammal host may be a human patient or an animal.

When the micelles are loaded into the nanogel, the amphiphilic molecule, particularly TGPS surprisingly reduce bacterial adhesion on the surface and does not prevent the pharmacological effect of the bioactive agent, therapeutic molecule or drug.

Such anti-adhesion effect is synergistically enhanced when a bacteriostatic agent is present into the nanogel.

In a fourth aspect, the present invention provides a method of producing a medical device, a biomaterial implant or a bioprosthesis with a nanogel coated surface comprising one or more bioactive molecule, therapeutic molecule or drug together with an amphiphilic molecule, preferably a vitamin-E derivative or an amphiphilic cyclodextrin; comprising the following-in-sequence steps of:
ci) optionally dipping the surface to be coated in a buffer solution of dopamine;
cii) dipping the surface coated at step ci) into a solution of polymer bearing primary or secondary amine groups; then
ciii) dipping the resulting coated surface obtained at step cii) into a liquid suspension of crosslinked-nanogel comprising one or more bioactive molecule, therapeutic molecule or drug together with the amphiphilic molecule, preferably the vitamin-E derivative or an amphiphilic cyclodextrin; said crosslinked-nanogel being obtained by one of both methods of the invention in liquid suspension preferably in water; then
civ) drying the crosslinked coated surface obtained at step ciii) to obtain a coated crosslinked-nanogel surface comprising one or more bioactive molecule, therapeutic molecule or drug together with the amphiphilic molecule, preferably the vitamin-E derivative or an amphiphilic cyclodextrin;
cv) optionally repeating steps cii) to civ) to obtain a surface coated with several layers of crosslinked nanogels comprising one or more bioactive molecule, therapeutic molecule or drug together with the amphiphilic molecule, preferably the vitamin-E derivative or an amphiphilic cyclodextrin;
Alternatively, the present invention also provides a method of producing a medical device, a biomaterial implant or a bioprosthesis with a crosslinked monolayer or optional multilayers coated surface comprising one or more bioactive molecule, therapeutic molecule or drug together with an amphiphilic molecule, preferably a vitamin-E derivative or an amphiphilic cyclodextrin; comprising the following-in-sequence steps of:
di) optionally dipping the surface to be coated in a buffer solution of dopamine;
dii) dipping the surface coated at step di) into a solution of polymer bearing primary or secondary amine groups; then
diii) dipping the resulting coated surface obtained at step dii) into a mixture of a poly(methacrylamide)-bearing quinone groups of formula (1)

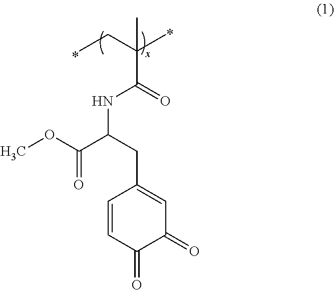

wherein x is an integer >1, preferably in the range between 1 and 100
with one or more bioactive molecule, therapeutic molecule or drug together with an amphiphilic molecule, preferably a vitamin-E derivative or an amphiphilic cyclodextrin;
dlv) drying the crosslinked coated surface obtained at step (diii) to obtain a coated crosslinked monolayer surface comprising one or more bioactive molecule, therapeutic molecule or drug together with the amphiphilic molecule, preferably the vitamin-E derivative or an amphiphilic cyclodextrin;
dv) optionally repeating steps dii) to div) to obtain a surface coated with crosslinked multilayers comprising one or more bioactive molecule, therapeutic molecule or drug together with the amphiphilic molecule, preferably the vitamin-E derivative or an amphiphilic cyclodextrin.

In optional step ci) and di), the medical device, biomaterial implant or bioprosthesis is first immersed in a buffer solution, particularly a Tris buffer solution with dopamine to strongly anchor a first polymer layer at the surface of the medical device, biomaterial implant or bioprosthesis. A primer coating of polydopamine PDA is then generated at the surface of the medical device, by polymerization of dopamine molecule having a 4-(2-aminoethyl) benzene-1,2-diol motif.

In step cii) and dii) the polymer bearing primary or secondary amine groups is preferably PAH and covalent grafting of PAH on the primer coating occurs through amine/quinone reaction and/or Schiff base formation.

In step diii) the precoated surface obtained at step dii) is dipped into a solution containing a poly(methacrylamide)-bearing quinone groups of formula (1)
mixed with one or more bioactive molecule, therapeutic molecule or drug together with an amphiphilic molecule, preferably a vitamin-E derivative or an amphiphilic cyclodextrin. Covalent grafting of the polymer layer is performed through the same reaction and/or Schiff base formation between primary or secondary amines, preferably from PAH monolayer and the quinone groups of poly(methacrylamide of formula (1)

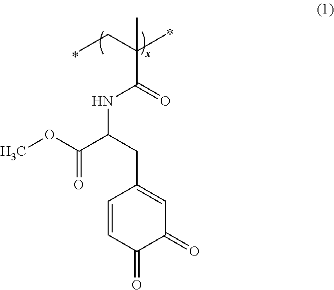

wherein x is an integer >1, preferably x is between 1 and 100;
In step ciii) the precoated surface obtained at step cii) is dipped into a liquid suspension of crosslinked-nanogel comprising one or more bioactive molecule, therapeutic molecule or drug together with an amphiphilic molecule, preferably a vitamin-E derivative or an amphiphilic cyclodextrin; said liquid suspension being preferably water.

In step cii) to cv), covalent grafting with the polymer bearing primary or secondary amine groups, preferably PAH occurs between quinone group-bearing layers coated on medical device, a biomaterial implant or a bioprosthesis resulting in a crosslinked nanogel coating on medical device, biomaterial implant or bioprosthesis;
In step dii) to dv), covalent grafting with the polymer bearing primary or secondary amine groups, preferably PAH occurs between monolayers coated on medical device, a biomaterial implant or a bioprosthesis resulting in a crosslinked multilayers coating on medical device, biomaterial implant or bioprosthesis

Similarly with poly(vinyl quinone), the present invention also provides a method of producing a medical device, a biomaterial implant or a bioprosthesis with a nanogel coated surface comprising one or more bioactive molecule, therapeutic molecule or drug together with an amphiphilic molecule, preferably a vitamin-E derivative or an amphiphilic cyclodextrin; comprising the following-in-sequence steps of:
ei) optionally dipping the surface to be coated in a buffer solution of dopamine;
eii) dipping the surface coated at step ei) into a solution of polymer bearing primary or secondary amine groups; then
eiii) dipping the resulting coated surface obtained at step eii) into a liquid suspension of crosslinked-nanogel comprising one or more bioactive molecule, therapeutic molecule or drug together with the amphiphilic molecule, preferably the vitamin-E derivative or an amphiphilic cyclodextrin, said crosslinked-nanogel being obtained by one of both methods of the invention in liquid suspension preferably in water;
eiv) drying the crosslinked coated surface obtained at step (eiii) to obtain a coated crosslinked-nanogel surface comprising one or more bioactive molecule, therapeutic molecule or drug together with the amphiphilic molecule, preferably the vitamin-E derivative or an amphiphilic cyclodextrin;
ev) optionally repeating steps eii) to eiv) to obtain a surface coated with several layers of crosslinked-nanogels comprising one or more bioactive molecule, therapeutic molecule or drug together with the amphiphilic molecule, preferably an vitamin-E derivative or an amphiphilic cyclodextrin;

Alternatively, the present invention also provides a method of producing a medical device, a biomaterial implant or a bioprosthesis with an optional multilayers coated surface comprising the steps of:
fi) optionally dipping the surface to be coated in a buffer solution of dopamine;
fii) dipping the surface coated at step fi) into a solution of polymer bearing primary or secondary amine groups; then
fiii) dipping the resulting coated surface obtained at step fii) into a mixture of a of poly(vinylquinone) of formula (6)

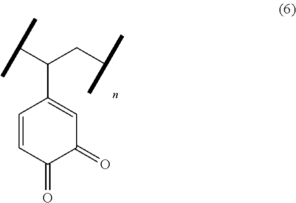

wherein n is an integer >1, preferably between 1 and 100
with one or more bioactive molecule, therapeutic molecule or drug together with an amphiphilic molecule, preferably a vitamin-E derivative or an amphiphilic cyclodextrin;
fiv) drying the crosslinked coated surface obtained at step (fiii) to obtain a crosslinked monolayer coated surface comprising one or more bioactive molecule, therapeutic molecule or drug together with the amphiphilic molecule, preferably the vitamin-E derivative or an amphiphilic cyclodextrin.
fv) optionally repeating steps fii) to fiv) to obtain a crosslinked multilayers coated surface comprising one or more bioactive molecule, therapeutic molecule or drug together with the amphiphilic molecule, preferably the vitamin-E derivative.

In step ei) and fi), the medical device, biomaterial implant or bioprosthesis is first immersed in a buffer solution, particularly a Tris buffer solution with dopamine (DOPA) to strongly anchor the first layer of the coating at the surface of the medical device, biomaterial implant or bioprosthesis. A primer coating of PDA is then generated at the surface of the medical device, by polymerization of dopamine molecule having 4-(2-aminoethyl) benzene-1,2-diol motif.

In step eii) and fii) the polymer bearing primary or secondary amine groups is PAH and covalent grafting of PAH on the primer coating occurs through amine/quinone reaction and/or Schiff base formation at room temperature.

In step fiii) the precoated surface obtained at step fii) is dipped into a solution containing a poly(vinylquinone) of formula (6) mixed with one or more bioactive molecule, therapeutic molecule or drug together with an amphiphilic molecule, preferably a vitamin-E derivative or an amphiphilic cyclodextrin. Covalent grafting of the polymer layer was performed through the same reaction and/or Schiff base formation between primary amines of PAH monolayer and the quinone groups of poly(vinylquinone) of formula (6).

In step eii) to ev), covalent grafting with the polymer bearing primary or secondary amine groups, preferably PAH occurs between nanogel layers coated on medical device, biomaterial implant or bioprosthesis resulting in a crosslinked nanogel coating on medical device, a biomaterial implant or a bioprosthesis.

In step fii) to fv), covalent grafting with the polymer bearing primary or secondary amine groups, preferably PAH occurs between monolayers coated on medical device, biomaterial implant or bioprosthesis resulting in a crosslinked multilayers coating on medical device, a biomaterial implant or a bioprosthesis.

Alternatively, the present invention also provides a method of producing a medical device, a biomaterial implant or a bioprosthesis with a coated surface comprising nanogel made of poly-(methacrylamide)-bearing quinone of formula (1) and polyvinylquinone of formula (6) or the copolymer thereof.

The present invention also extends to methods of producing a biomaterial implant, medical device or bioprosthesis with a surface coated with nanogel made of polymers or copolymers bearing quinone groups.

The obtained nanogel may comprise one or more, preferably two or more bioactive molecules, therapeutic molecules and/or drugs. The bioactive molecules may include an antibiotic and/or an anti-platelet agent.

The method of the invention may be used without the need of a primer coating step. In this case, coating adhesion is based on adhesive properties of the free quinone groups present at the nanogel surface, which is sufficient to coat and immobilized the nanogel according to the invention at the surface of the medical device, biomaterial implant or bioprosthesis.

A medical device, a biomaterial implant or a bioprosthesis with a surface coated with a two- or more layer-crosslinked nanogel may be produced by repeating steps cii) and ciii) or eii) and eiii) of the above-described methods. A medical device, a biomaterial implant or a bioprosthesis comprising 2, 3, 4, 5 or more layers of nanogel may be produced.

In a preferred embodiment, the bioactive molecule, therapeutic molecule or drug is a triazolo(4,4-d)-pyrimidine derivative of formula (3); preferably
(1S,2S,3R,5S)-3-[7-[(1R,2S)-2-(3,4-difluorophenyl)cyclopropylamino]-5-(propylthio)-3H-[1,2,3]-triazolo[4,5-d]pyrimidin-3-yl]-5-(2-hydroxyethoxy)-1,2-cyclopentanediol) also called Triafluocyl or ticagrelor; or
is (1S,2R,3S,4R)-4-[7-[[(1R,2S)-2-(3,4-Difluorophenyl)-cyclopropyl]amino]-5-(propylthio)-3H-1,2,3-triazolo[4,5-d]pyrimidin-3-yl]-1,2,3-cyclopentanetriol also called Fluometacyl or Fluometacyl® that have both anti-bacterial and antiplatelet properties.

In another preferred embodiment the triazolo(4,4-d)-pyrimidine derivative is combined with a bacteriostatic antibiotic, preferably minocycline. The triazolo(4,4-d)-pyrimidine derivative is preferably Triafluocyl or Fluometacyl or Fluometacyl®.

In another preferred embodiment the bioactive molecule, therapeutic molecule or drug is a bisguanide, most preferably chlorhexidine.

The method of the invention may be used to coat just a part of the surface of a medical device, a biomaterial implant or a bioprosthesis, or substantially the whole or the whole surface of a medical device, a biomaterial implant or a bioprosthesis.

The invention further provides a coated medical device, a biomaterial implant or a bioprosthesis according to the invention or produced by the method of the invention for use in the prevention or reduction of infection when the medical device, a biomaterial implant or a bioprosthesis is implanted in a mammal that can be a human patient or an animal.

In a fifth aspect, the invention further provides a pharmaceutical composition comprising the nanogel according to the invention or produced by the method of the invention for use in the prevention or reduction of infection when apply to a host mammal by topical administration. The host mammal may be a human patient or an animal.

In a preferred embodiment the pharmaceutical composition comprising the nanogel according to the invention is applied on animal preferably dog, sheep or cattle for treatment of dermatosis generated by bacterial infection such as mastitis or pyoderma.

The pharmaceutical composition comprising the nanogel according to the invention is preferably a gel, but can also be a liquid composition having physiological compatibility.

The pharmaceutical compositions may include, in addition to the bioactive molecule, therapeutic molecule or drug together with the amphiphilic molecule; auxiliary substances, preservatives, solvents and/or viscosity modulating agents. By solvent, one means for example water, saline or any other physiological solution, ethanol, glycerol, oil such as vegetable oil or a mixture thereof. By viscosity modulating agent on means for example carboxymethylcellulose.

In a preferred embodiment, the bioactive molecule, therapeutic molecule or drug is an anti-infective agent or a bactericidal agent or an antivirulence agent together with a bacteriostatic agent, preferably minocycline or chlorexhidine

In a most preferred embodiment, the bioactive molecule, therapeutic molecule or drug is Triafluocyl.

In a sixth aspect, the invention also provides the uses of the nanogel according to the invention or produced by a method of the invention for inhibiting bacterial adhesion on a surface of medical device, particularly the surface of a catheter.

The method of inhibiting bacterial adhesion on a surface may comprise the following steps:
i) optionally dipping the surface to be inhibited of bacterial adhesion in a buffer solution of dopamine;
ii) dipping the surface optionally coated at step i) into a solution of polymer bearing primary or secondary amine groups;
iii) dipping the surface obtained at step ii) into a solution of a poly(methacrylamide)-bearing quinone groups of formula (1) mixed with one or more bioactive molecule, therapeutic molecule or drug together with an amphiphilic molecule, preferably a vitamin-E derivative or an amphiphilic cyclodextrin;
iv) drying the crosslinked coated surface obtained at step (iii) to obtain a coated crosslinked monolayer surface comprising one or more bioactive molecule, therapeutic molecule or drug together with the amphiphilic molecule, preferably the vitamin-E derivative or an amphiphilic cyclodextrin.

Alternatively, the method of inhibiting bacterial adhesion on a surface may comprise the following steps:
i) dipping the surface to be inhibited of bacterial adhesion in a buffer solution of dopamine;
ii) dipping the surface optionally coated at step i) into a solution of polymer bearing primary or secondary amine groups;
iii) dipping the surface obtained at step ii) into a solution of a poly(vinyl quinone) of formula (6) mixed with one or more bioactive molecule, therapeutic molecule or drug together with an amphiphilic molecule, preferably a vitamin-E derivative or an amphiphilic cyclodextrin;
iv) drying the crosslinked coated surface obtained at step (iii) to obtain a coated crosslinked monolayer surface comprising one or more bioactive molecule, therapeutic molecule or drug together with the amphiphilic molecule, preferably the vitamin-E derivative or an amphiphilic cyclodextrin.

Alternatively, the method of inhibiting bacterial adhesion on a surface may comprise the following steps:
i) optionally dipping the surface to be inhibited of bacterial adhesion in a buffer solution of dopamine;
ii) dipping the surface coated at step i) into a solution of polymer bearing primary or secondary amine groups; then
iii) dipping the resulting coated surface obtained at step ii) into a liquid suspension of crosslinked-nanogel comprising one or more bioactive molecule, therapeutic molecule or drug together with the amphiphilic molecule, preferably the vitamin-E derivative or an amphiphilic cyclodextrin; said crosslinked-nanogel being obtained by one of both methods of the invention in liquid suspension preferably in water; then
iv) drying the crosslinked coated surface obtained at step (iii) to obtain a coated crosslinked-nanogel surface comprising one or more bioactive molecule, therapeutic molecule or drug together with the amphiphilic molecule, preferably the vitamin-E derivative or an amphiphilic cyclodextrin;
v) optionally repeating steps cii) to civ) to obtain a surface coated with several layers of crosslinked nanogels comprising one or more bioactive molecule, therapeutic molecule or drug together with the amphiphilic molecule, preferably the vitamin-E derivative or an amphiphilic cyclodextrin;

The invention will be further described by means of non-limiting examples only, with reference to the following figures and experimental examples.

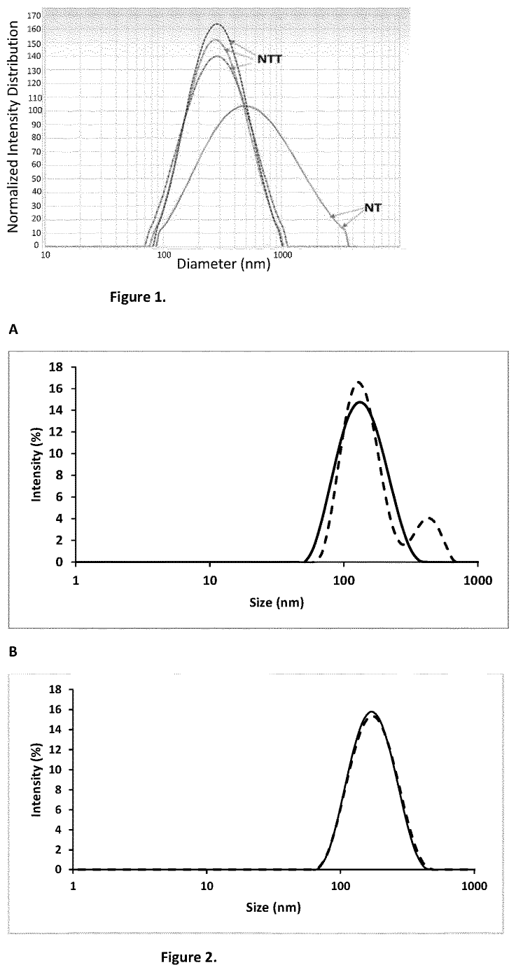

FIG. 1 shows dynamic size analysis (dynamic light scattering, DLS) of nanogels diameter in water suspension NTT comprising Triafluocyl and TPGS according to the invention compared to nanogels NT comprising triafluocyl only according to WO2018/122318.

FIG. 2 shows nanogels diameter in water suspension comprising triafluocyl alone (A) or in presence of TPGS (B) according to the present invention. Solid line: DLS analysis realized 1 h after nanogel formation. Dashed line: DLS analysis realized after 24 h.

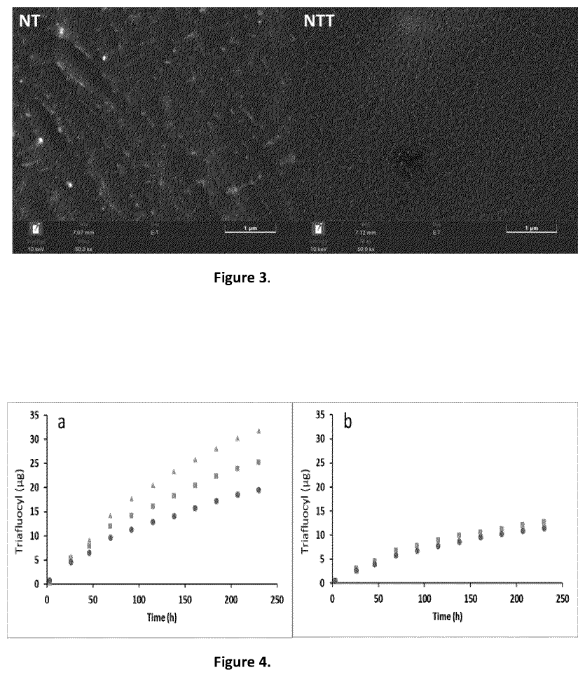

FIG. 3 shows surface topography, by a SEM analysis, of a Polyurethane (PU) catheter coated with a nanogel according to the invention (NTT) compared to surface topography of a PU catheter coated with a nanogel (NT) according to WO2018/122318

FIG. 4 shows kinetic release of Triafluocyl from a nanogel (NTT) according to the invention compared to the kinetic release of Triafluocyl from a nanogel (NT) according to WO2018/122318.

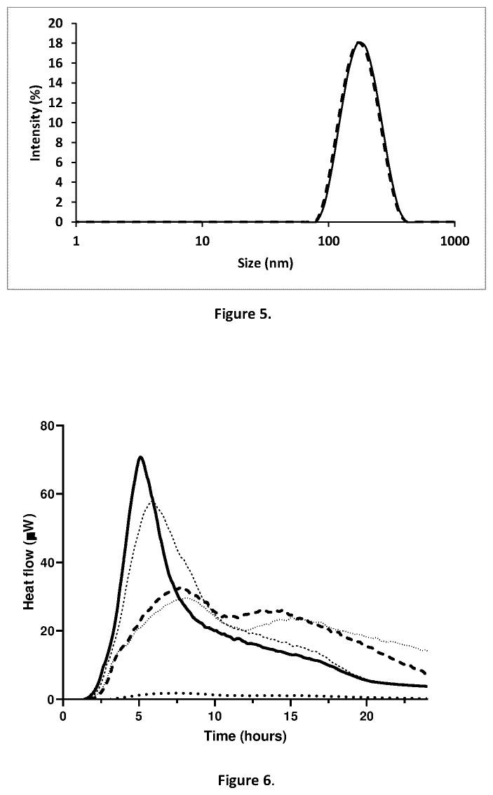

FIG. 5 shows nanogels diameter in water suspension comprising triafluocyl, TPGS and minocycline. Solid line: DLS analysis realized 1 h after nanogel formation. Dashed line: DLS analysis realized after 24 h.

FIG. 6 shows real-time microcalorimetric measurements of S. aureus BAA-1556 metabolic activity expressed as the heat flow. Solid line stands for control bacteria with a vehicle (0.6% ethanol); thick dashed line—bacteria treated with 10 μg/mL of triafluocyl; thick dotted line—bacteria treated with 20 μg/mL of triafluocyl; thin dashed line—bacteria treated with both triafluocyl and TPGS (10 μg/mL:10 μg/mL); thin dotted line—bacteria treated with both triafluocyl and TPGS (20 μg/mL:20 μg/mL).

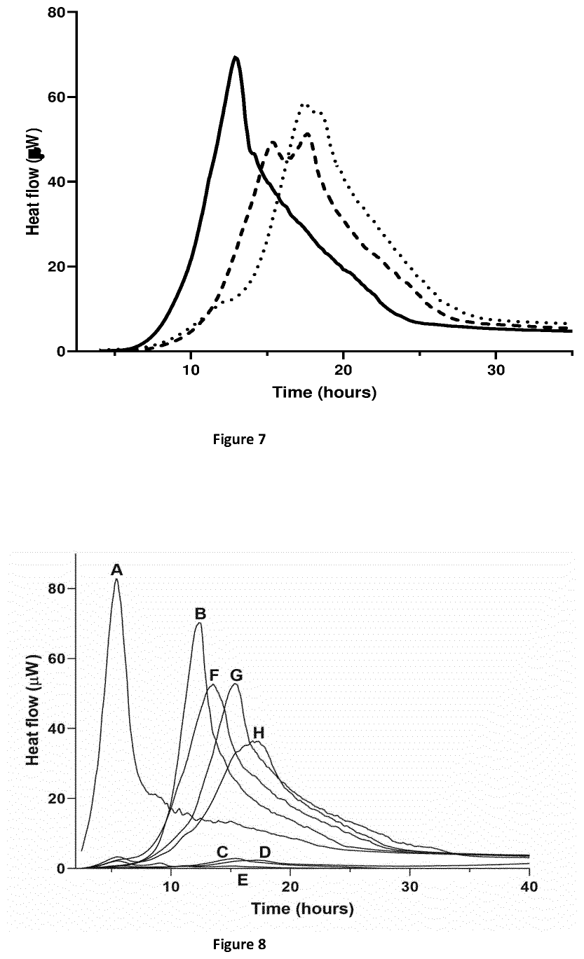

FIG. 7 shows real-time microcalorimetric measurements of S. aureus BAA-1556 metabolic activity upon adhesion to catheters, expressed as the heat flow. Solid line—control non-coated PU catheter; dashed line—PU catheter coated with triafluocyl-loaded nanogel; dotted line—PU catheter coated with triafluocyl-TPGS-loaded nanogel.

FIG. 8 shows real-time microcalorimetric measurements of bacterial metabolic activity upon adhesion to catheters, expressed as the heat flow. A—Baseline growth of S. aureus BAA-1556 (MRSA) at the dilution of 10⁻⁶ (no catheter segments); B—control non-coated catheter segment; C—NGM—catheter segment coated with the 5 layers of nanogel loaded with minocycline 0.5 mg/mL added in the last nanogel layer and grafted with PEG (t=0 days); D—NTM—catheter segment coated with the 5 layers of nanogel loaded with ticagrelor 0.05 mg/mL and followed by minocycline (0.5 mg/mL) added to the last layer of nanogel and grafted with PEG (t=0 days); E—NTTM—catheter segment coated with the 5 layers of nanogel loaded with the combination of Triafluocyl®/TPGS (0.1/0.2 mg/mL, w/w) and followed by minocycline (0.5 mg/mL) added to the last layer of nanogel (t=0 days); F—NGM—catheter segment coated with the 5 layers of nanogel loaded with minocycline 0.5 mg/mL added in the last nanogel layer and grafted with PEG (t=20 days); G—NTM—catheter segment coated with the 5 layers of nanogel loaded with Triafluocyl® 0.05 mg/mL followed by minocycline (0.5 mg/mL) added to the last layer of nanogel and grafted with PEG (t=20 days); H—NTTM—catheter segment coated with the 5 layers of nanogel loaded with the combination of Triafluocyl®/TPGS (0.1/0.2 mg/mL, w/w) followed by minocycline (0.5 mg/mL) added to the last layer of nanogel (t=20 days).

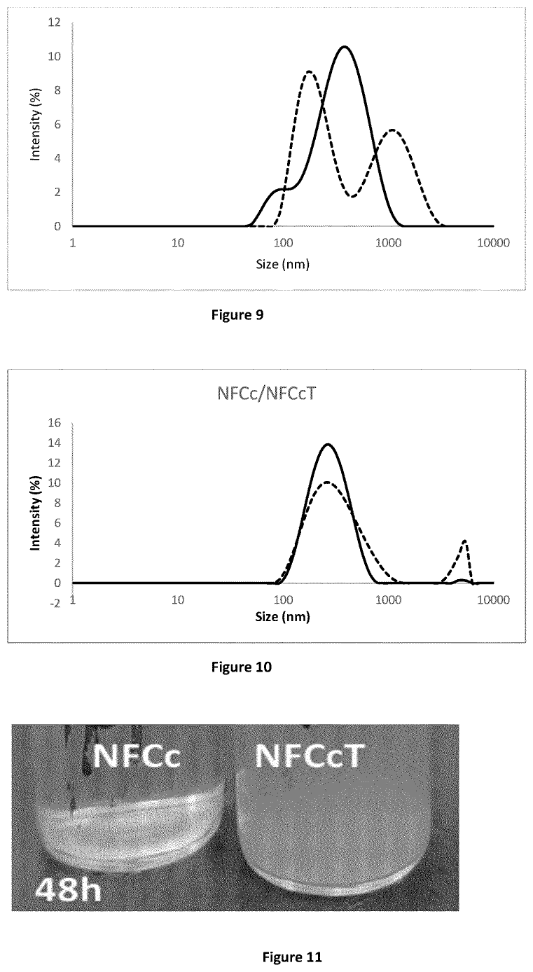

FIG. 9 shows DLS analysis of nanogel diameter in water suspension comprising chlorhexidine chloride (Chc) and TPGS in solid line, and without TPGS in dashed line.

FIG. 10 shows DLS analysis of nanogel diameter in water suspension comprising triafluocyl, chlorhexidine (Chc) and TPGS in solid line and without TPGS in dashed line.

FIG. 11 shows photography of nanogel with Fluometacyl®, Chc and TPGS (NFCcT) and without TPGS (NFCc) showing complete precipitation of NFCc after 48 hours.

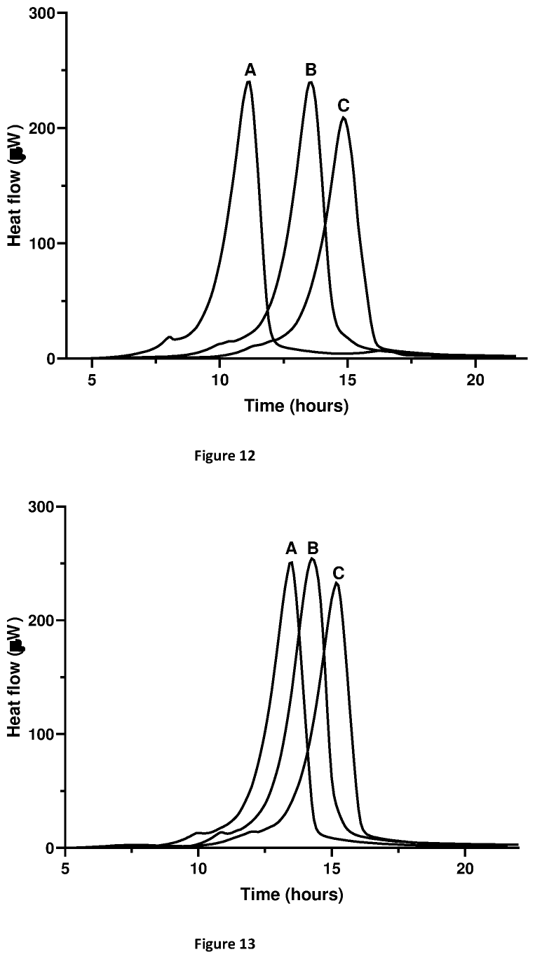

FIG. 12 shows the real-time microcalorimetric measurements of bacterial metabolic activity upon adhesion to catheters, expressed as the heat flow. Curve A—control bacterial growth for P. aeruginosa 10⁻⁷; Curve B—Bacteria with the nanogel-coated PU catheter loaded with Fluometacyl® and chlorhexidine chloride (NFCc); Curve C—Bacteria with the nanogel-coated PU catheter loaded with Fluometacyl® and chlorhexidine chloride in the presence of TPGS (NFCcT).

FIG. 13 shows the real-time microcalorimetric measurements of P. aeruginosa metabolic activity upon adhesion to catheters, expressed as the heat flow. Curve A—Bacteria with the nanogel-coated PU catheter loaded with Fluometacyl® and chlorhexidine chloride (NFCc); Curve B—Bacteria with the nanogel-coated PU catheter loaded with Fluometacyl® and soaked in the solution of chlorhexidine chloride (1 mg/mL) (NF/Cc); Curve C—Bacteria with the nanogel-coated PU catheter loaded with Fluometacyl® and chlorhexidine chloride in the presence of TPGS (NFCcT).

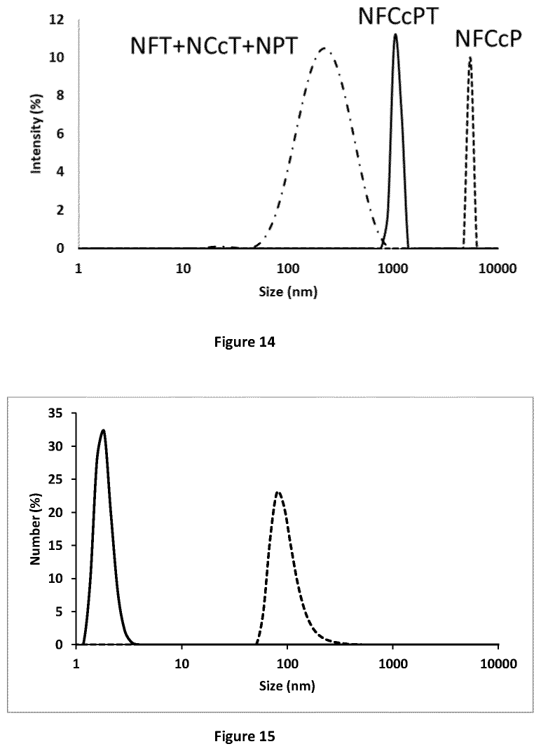

FIG. 14 shows DLS analysis of nanogel diameter in water suspension, the nanogel comprising mixture of Fluometacyl, Chlorhexidine and Polymyxin B (NFCccP).

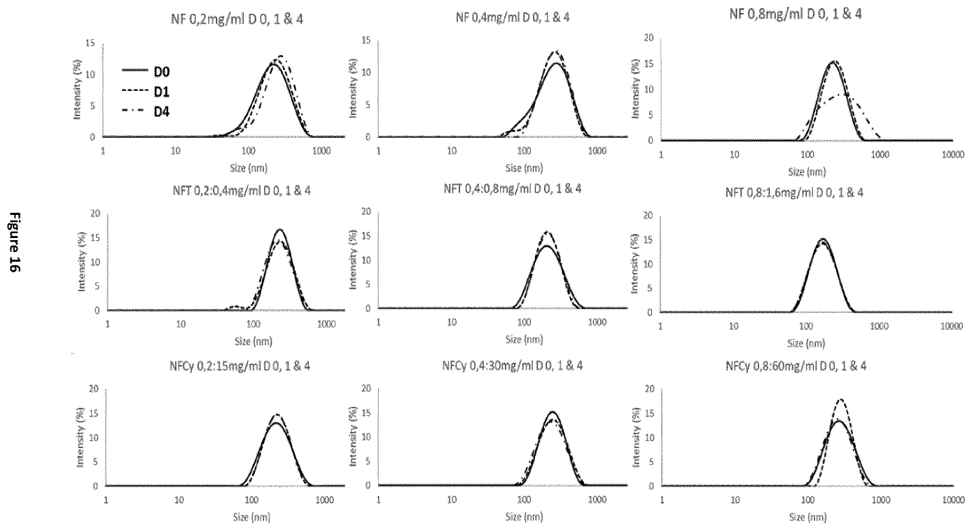

FIG. 15 shows DLS analysis of nanogel diameter in water suspension, the nanogel comprising Fluometacyl and Cyclodextrin (NFCy) before (solid line) and after addition of PAH (Dashed line).

FIG. 16 shows stability study of nanogel diameter in water suspension for NF, NFT and NFCy with different concentration of fluometacyl and stabilizer (TPGS or cyclodextrin) using DLS analysis.

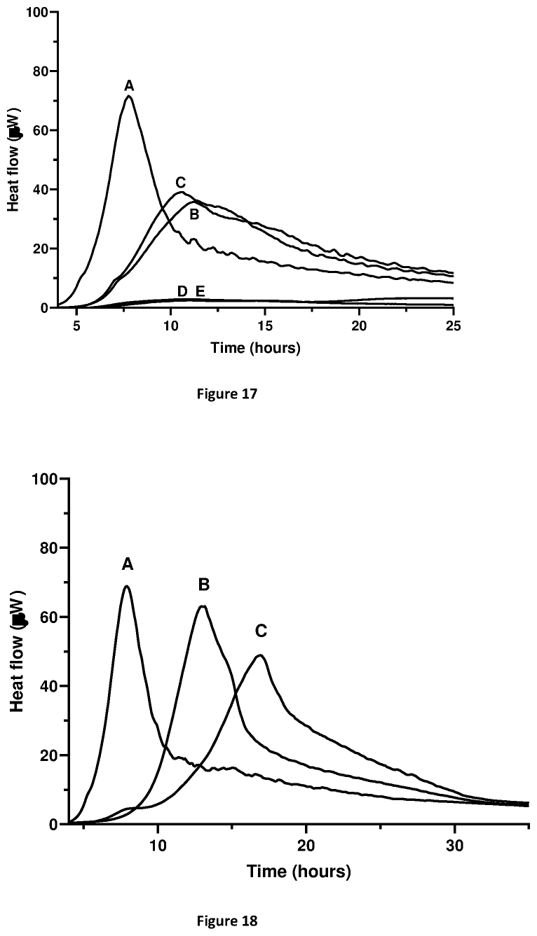

FIG. 17 shows the real-time microcalorimetric measurements of bacterial metabolic activity expressed as the heat flow. Curve A—control bacterial growth for S. aureus MRSA 10⁻⁶; Curve B—Bacteria with Fluometacyl® (10 μg/mL); Curve C—Bacteria with Fluometacyl® (10 μg/mL) and hydroxypropyl-β-cyclodextrin (42 g/mL); Curve D—Bacteria with fluometacyl (20 μg/mL); Curve E—Bacteria with Fluometacyl® (20 μg/mL) and hydroxypropyl-β-cyclodextrin (42 μg/mL).

FIG. 18 shows the real-time microcalorimetric measurements of bacterial metabolic activity upon adhesion to catheters, expressed as the heat flow. Curve A—control bacterial growth for S. aureus MRSA 10⁻⁶; Curve B—Bacteria with the nanogel-coated PU catheter loaded with Fluometacyl® and TPGS (0.2/0.4 mg/ml, w/w); Curve C—Bacteria with the nanogel-coated PU catheter loaded with Fluometacyl® and hydroxypropyl-β-cyclodextrin (0.2/15 mg/mL, w/w).

A Table of references is reported hereafter summarizing different nanogel composition obtained according to the invention in the following examples:
NT nanogel according to WO 2018/122318A1
NTT nanogel with triafluocyl and TPGS
NG nanogel without bioactive molecule, therapeutic molecule or drugs
NT nanogel with triafluocyl
NTTM nanogel with triafluocyl, minocycline and TPGS
NCcT nanogel with chlorhexidine chloride and TPGS
NFCcT nanogel with Fluometacyl®, chlorhexidine Chloride and TPGS
NFCc nanogel with Fluometacyl®, chlorhexidine chloride
NFCy nanogel with Fluometacyl and hydroxypropyl-beta-cyclodextrin.
NF nanogel with Fluometacyl
NFT nanogel with Fluometacyl and TPGS
NFCccP nanogel with Fluometacyl, Chlorhexidine and Polymixin B

### Example 1: Preparation of the Nanogel According to the Invention with P(mDOPA) Triafluocyl and TPGS (Also Called NTT)

The nanogel preparation is similar to the one disclosed in WO2018/122318A1, excepted for drug loading in presence of an additional amphiphilic molecule.

The Nanogel is prepared in liquid solution. After crosslinking the nanogel remains suspended in the liquid solution.

1.1 oxidation of PmDOPA→Pox(mDOPA).

A homopolymer of methacrylamide bearing 3,4-dihydroxy-L-phenylalanine (P(mDOPA) is synthetised according to Faure & al in Adv Funct. Mater. 2012; 22:5271-5282, and is oxidized in aqueous media under basic conditions for 12 hours to form hydrosoluble Pox(mDOPA). Oxidized catechol moieties of Pox(mDOPA) are necessary for the covalent interaction of PAH through amine/quinone reaction and/or Schiff base formation at room temperature, and consequently for the preparation of stable cross-linked nanogel in water suspension.

P(mDOPA) (2.5 mg) was dissolved in distilled water (5 mL) and NaOH (0.1 M) was slowly added in order to raise the pH above 10 and to promote the oxidation of catechol groups of P(mDOPA).

1.2 Preparation of Triafluocyl/TPGS Mixture in Ethanol Triafluocyl (also called Ticagrelor and provided by Polpharma) and TPGS provided by MedChemExpress LLC were dissolved separately in ethanol to prepare stocks solution of 3.33 mg/mL. 160 μL of triafluocyl solution and 160 μl of TPGS solution were mixed under stirring (300 rpm) with a magnetic stirrer and then concentrated to about 160 μl under vacuum at RT.
1.3 Preparation of the Nanogel with Triafluocyl/TPGS and PAH

Pox(mDOPA) (5 ml, 0.5 mg/ml) was added under stirring (300 rpm) with a magnetic stirrer, to the concentrated mixture obtained at point 1.2. After one hour of homogenization at RT, an aqueous solution of PAH provided from Sigma Aldrich (0.5 mL, 0.5 mg/ml) at pH 10 was slowly added to the mixture solution. The solution was allowed to react for one hour at room temperature under vigorous stirring (500 rpm) with a magnetic stirrer.

Nanogels with a diameter ranging from 100 nm to 350 nm were observed in water suspension by Dynamic Light Scattering (Zetasizer Advance Pro, Malvern).

### Example 2: Preparation of the Nanogel According to the Invention with Polyvinyl(Quinone), Triafluocyl and TPGS

### 2.1 Oxidation of Polyvinyl Catechol→Polyvinylquinone).

Similarly to P(mDOPA), poly(vinylcatechol) provided by Polykey is oxidized in aqueous media under basic conditions for 12 hours to form watersoluble Polyvinylquinone. Oxidized catechol moieties of Poly(vinylcathechol) are necessary for the covalent interaction of PAH through amine/quinone reaction and/or Schiff base formation at room temperature, and consequently for the preparation of stable cross-linked nanogel in water suspension.

Poly(vinylcathechol) (10 mg) was dissolved in distilled water (20 mL) and NaOH (0.1 M) was slowly added in order to raise the pH above 10 for 12 hours and to promote the oxidation of catechol groups of Poly(vinylcatechol).

### 2.2 Preparation of Triafluocyl/TPGS Mixture in Ethanol

Triafluocyl (also called Ticagrelor) and TPGS were dissolved separately in ethanol to prepare stocks solution of 3.33 mg/mL triafluocyl and of TPGS. 160 μL of triafluocyl solution and 160 μl of TPGS solution were mixed under stirring (300 rpm) with a magnetic stirrer and then concentrated to about 160 μl under vacuum at RT.

2.3 Preparation of the Nanogel with Triafluocyl/TPGS and PAH

Poly(vinylquinone) (5 ml, 0.5 mg/ml) was added under stirring (300 rpm) with a magnetic stirrer to the concentrated mixture obtained at point 2.2. After one hour of homogenization, an aqueous solution of PAH (0.5 mL, 0.5 mg/ml) at pH was slowly added to the mixture solution. The solution was allowed to react for one hour at room temperature under vigorous stirring (500 rpm) with a magnetic stirrer.

Nanogel with a diameter ranging from 100 nm to 350 nm were observed in water suspension by Dynamic Light Scattering (Zetasizer Advance Pro, Malvern).

### Example 3: Preparation of a Medical Device with a Nanogel According to the Invention

A polyurethane catheter provided from Carfill (intravascular grade polyurethane tubing 5 Fr), was coated according to the method of the invention.

In Step 1: catheters (4.5 cm long) were dipped into a Dopamine Tris buffer solution pH=7.4 from Aldrich (0.2 g L-1) for 5 h.

In Step 2: After rinsing twice with 5 ml water, the modified catheter substrate was dipped into an aqueous solution of PAH (pH>10) for 1 h and rinsed twice with 5 ml water.

In step 3: The modified catheter substrate obtained in step 2 was dipped into an aqueous solution of the bioactive molecule-loaded nanogels prepared according to example 1 or 2 for 18 h and rinsed twice with 5 ml water.

Steps 2 to 3 were repeated to build-up a multilayer assembly of nanogels on the surface of coated device (five times to obtain a five-layer assembly of cross-linked nanogels).

### Example 4: Comparison of the Nanogel NTT According to the Invention to the Nanogel NT of WO2018/122318A1

To allow a comparison with the nanogel of the present invention, a nanogel with and without triafluocyl are prepared according to WO2018/122318A1.

Nanogel Preparation (NG) without Triafluocyl:

P(mDOPA) (2.5 mg) was dissolved in distilled water (5 mL) and NaOH (0.1 M) was slowly added in order to raise the pH above 10 and to promote the oxidation of catechol groups of P(mDOPA). After one night at room temperature, an aqueous solution of PAH (0.5 mL; 0.5 g/L) at pH 10 was slowly added to the solution of Pox(mDOPA). The solution was allowed to react for one hour at room temperature under vigorous stirring (500 rpm with a magnetic stirrer). Nanogel with a diameter ranging from 100 nm to 250 nm were observed in water suspension by Dynamic Light Scattering (Zetasizer Advance Pto, Malvern).

Nanogel Preparation with Triafluocyl (NT):

P(mDOPA) (2.5 mg) was dissolved in distilled water (5 mL) and NaOH (0.1 M) was slowly added in order to raise the pH above 10 and to promote the oxidation of catechol groups of P(mDOPA). After one night at room temperature, 0.16 ml of Triafluocyl solution (3.33 mg/ml in DMSO) was added dropwise to the solution of Pox(mDOPA) under stirring (300 rpm with a magnetic stirrer) at room temperature. After one hour of homogenization, an aqueous solution of PAH (0.5 mL; 0.5 g/L) at pH 10 was slowly added to the mixture solution. The solution was allowed to react for one hour at room temperature under vigorous stirring (500 rpm with a magnetic stirrer). Nanogel NT with a diameter ranging from 120 nm to 750 nm were observed in water suspension by Dynamic Light Scattering (Zetasizer Advance Pro, Malvern).

### 4.1. Particle Size and Stability

The nanogel particle diameter size according to the invention is compared to the one obtained for the nanogel (NT) of WO2018/122318A1 with and without triafluocyl.

Compared to the nanogel diameter without Triafluocyl and TPGS (ranging from 100 nm to 250 nm) and to the nanogel with Triafluocyl only (ranging from 120 nm to 750 nm, the present nanogel diameter (ranging from 100 nm to 350 nm) is clearly intermediate between a nanogel particle alone and a nanogel particle comprising Triafluocyl. The present nanogel invention shows also a lower size distribution as illustrated in FIG. 1.

FIG. 2 shows increased size of nanogel NT after 24 h (dashed line), depicting nanogel aggregate formation or precipitation, which was not observed for NTT nanogel that remained stable after 24 h.

Consequently, the nanogel according to the invention are more stable than the one disclosed in WO2018/122318A1

### 4.2 Surface Topography

As can be seen from FIG. 3, the SEM analysis of the coated catheters showed an obvious difference between NT and NTT coatings. More homogeneous and smooth surface are observed with NTT coating whereas large particle aggregation present in NT coating are observed, which increases surface roughness.

### 4.3 Surface Hydrophilicity

Contact angle measurement is a useful method of determining the surface hydrophilicity. Table 1 reports contact angle analysis on glass cover slip (CS) provided by WTR. CS were coated with polydopamine (CS-PDA). one, three and five layers of NTT (CS-NTT 1 Layer, 3 Layers and 5 Layers) and compared with five layers of nanogel according to WO2018/122318A1 NT (CS-NT 5 LayersPEG). As in WO2018/122318A1, PEG (MW 2000 g/mol) was covalently linked on top of nanogel coating. Contact angles were measures after 30 seconds.

The static contact angles decreased obviously in the presence of nanogel layers (NT or NTT) compared to PDA (primer polydopamine). The coated surface with five layers of NTT (nanogel with TPGS) displays much lower water contact angles (18.3°) than the coated surface with same number of layers of NT (35.5°) indicating that the presence of PEG chains (1000 g/mol) in TPGS molecules improved the surface hydrophilicity significantly even after the deposition of only one layer of NTT (25.3°).

TABLE 1

| Contact | CS- | CS-NTT | CS-NTT | CS-NTT | CS-NT 5Layers |
|---|---|---|---|---|---|
| angle | PDA | 1Layer | 3Layers | 5Layers | PEG |
| | 60.3 | 25.3 | 24.6 | 18.3 | 35.5 |

### 4.3 Kinetic Release

The in vitro drug release from the coated catheters surface was quantified at defined time points up to 10 days. UV spectrophotometry was used to analyze the impact of the presence of TPGS on drug release kinetics and drug release profiles were created for each catheter from the experimentally obtained data (FIG. 4).

It was found that the presence of precipitates in NT coating has a significant impact on drug release kinetics of the coated catheters. The drug release data shows that both coatings showed a progressive and continuous release of the drug. However, a large variability was observed for the NT coating, depicting a lack of homogeneity. Moreover, the kinetics of release of triafluocyl with NT was much quicker than with NTT, resulting in shortened release duration, which will negatively impact the duration of coating pharmacological activity.

### 4.4 Triafluocyl Content in Coated Catheters

Six polyurethane 4.5-cm long catheters provided from Carfill (intravascular grade polyurethane tubing 5 Fr), were coated with NTT or NT according to the method of the invention (NTT) or to WO2018/122318A1 (NT).

Triafluocyl content in coated catheters (4.5 cm) was determined by HPLC on a Waters Acquity UPLC System consisting of a quaternary solvent delivery system, an injector with adjustable injection volume, temperature controlled autosampler, column thermostat and photo diode array detector. The assay method was used according to ticagrelor monograph (European Pharmacopeia 10.4). Briefly, the analytical column was a XBridge Phenyl, 150×4.6 mm, 3 μm (Waters) with the guard column Security Guard Phenyl, 3×4 mm (Phenomenex). An injection volume of 50 μL was used at a flow rate of 1.0 ml/min at 40° C. (column temperature). Mobile phase A: was phosphate buffer pH3.0-water-acetonitrile (1:89:10 v/v/v). Mobile phase B: phosphate buffer pH3.0-water-acetonitrile (1:29:70 v/v/v). Detection wavelength was 300 nm.

Data shown in Table 2 indicate that a two-fold higher amount of triafluocyl was obtained in NTT coated catheters as compared to catheters coated with NT.

TABLE 2

| | | Triaflucocyl content |
|---|---|---|
| Sample No. | Coating type | (μg/cm) |
| 1 | NT | 6.8 |
| 2 | NT | 5.0 |
| 3 | NT | 5.9 |
| 4 | NT | 4.8 |
| 5 | NT | 6.5 |
| 6 | NT | 5.4 |
| 7 | NTT | 12.0 |
| 8 | NTT | 9.5 |
| 9 | NTT | 11.3 |
| 10 | NTT | 9.8 |
| 11 | NTT | 12.9 |
| 12 | NTT | 11.2 |

### Example 5: Preparation of the Nanogel According to the Invention with P(mDOPA) Triafluocyl, Minocycline and TPGS (Also Called NTTM)

### 5.1 Oxidation of PmDOPA→Pox(mDOPA).

A homopolymer of methacrylamide bearing 3,4-dihydroxy-L-phenylalanine (P(mDOPA), is oxidized in aqueous media under basic conditions for 12 hours to form the hydrosoluble Pox(mDOPA). Oxidized catechol moieties of Pox(mDOPA) are necessary for the covalent interaction of PAH through amine/quinone reaction and/or Schiff base formation at room temperature, and consequently for the preparation of stable cross-linked nanogel in aqueous suspension. P(mDOPA) (2.5 mg) was dissolved in distilled water (5 mL) and NaOH (0.1 M) was slowly added in order to raise the pH above 10 and to promote the oxidation of catechol groups of P(mDOPA) into Pox(mDOPA).

### 5.2 Preparation of Triafluocyl/Minocycline/TPGS Mixture in Ethanol

Triafluocyl (also called Ticagrelor), minocycline and TPGS were dissolved separately in ethanol to prepare stock solutions of 3.33 mg/mL. 160 μL of triafluocyl solution, 320 μl of TPGS solution and 320 μl of minocycline solution were mixed under stirring (300 rpm) and then concentrated to about 160 μl under vacuum at RT.

5.3 Preparation of the Nanogel with Triafluocyl/Minocycline/TPGS and PAH

Pox(mDOPA) (5 ml, 0.5 mg/ml) was added under stirring to the concentrated mixture obtained at point 5.2. After one hour of homogenization at RT, an aqueous solution of PAH (0.5 mL, 0.5 mg/ml) at pH 10 was slowly added to the mixture solution. The solution was allowed to react for one hour at room temperature under vigorous stirring (500 rpm).

Stable nanogels with a diameter ranging from 150 nm to 350 nm were observed in water suspension by Dynamic Light Scattering (Malvern). (FIG. 5)

NTTM nanogel were stable up to 24 h after their formation as were NTT nanogels. In contrast, NT nanogel according to WO2018/122318A1 were not stable (Table 3).

TABLE 3

| | 1 h | 3 h | 5 h | 24 h |
|---|---|---|---|---|
| | Particle | Particle | Particle | Particle |
| | diameter | diameter | diameter | diameter |
| Nanogel type | (nm) | (nm) | (nm) | (nm) |
| NT | 126 | 128 | 132 | 400 |
| NTT | 160 | 160 | 161 | 162 |
| NTTM | 172 | 171 | 171 | 170 |

On the other hand, when triafluocyl is released from the nanogel prepared according to the method of the invention and therefore encapsulated by TPGS, less inhibiting effect of TPGS is surprisingly observed as illustrated in the following example.

### Example 6. Effect of TPGS on the Inhibitory Activity of Triafluocyl on Bacterial Growth

S. aureus (ATCC BAA-1556, MRSA) was grown overnight in TSB (tryptic soy broth) medium, before being diluted 1×10⁴ fold in fresh TSB. Subsequently 300 L aliquots of diluted bacteria suspensions were supplemented with 10 μg/mL and 20 μg/mL of respectively triafluocyl (Polpharma) alone or in the mixture with TPGS (MedChemExpress LLC (Europe)) (1:1 w/w). Both Triafluocyl and TPGS were prepared in absolute ethanol as already mentioned in the above examples and were refrigerated as master stocks in absolute ethanol. Triafluocyl, TPGS or ethanol (0.6% final conc.) as a vehicle were added to the bacterial suspensions to obtain the concentrations (10 μg/mL and 20 μg/m) followed by a brief vortexing. Bacterial aliquots were then distributed in the dedicated non-activated inserts of a 48-well plate and grown for 24 h under static conditions at 37° C. using Calscreener technology to measure bacterial growth and metabolic activity in Real-Time as described in https://cordis.europa.eu/project/id/784514.

In FIG. 6, the real-time microcalorimetric measurements of bacterial metabolic activity is expressed as the heat flow and represents bacterial growth in TSB medium. Solid line stands for control bacteria with a vehicle (0.6% ethanol); thick dashed line—bacteria treated with 10 μg/mL of triafluocyl; thick dotted line—bacteria treated with 20 μg/mL of triafluocyl; thin dashed line—bacteria treated with both triafluocyl and TPGS (10 μg/mL:10 μg/mL); thin dotted line—bacteria treated with both triafluocyl and TPGS (20 μg/mL:20 μg/mL).

Triafluocyl also called Ticagelor significantly reduces S. aureus bacteria growth at the concentration of 10 μg/mL, whereas at the higher dose of 20 μg/mL, bacterial growth was fully inhibited. Strikingly an addition of TPGS at the equivalent amount (w/w) to triafluocyl significantly reduced the inhibitory effect of triafluocyl on bacterial growth.

### Example 7 Testing Bacterial Anti-Attachment Property of Nanogel Coating with Triafluocyl Alone and Together with TPGS Against S. aureus

S. aureus (ATCC BAA-1556, MRSA) was grown overnight in TSB (tryptic soy broth) medium, before being diluted 1×10⁶ fold in fresh TSB. Subsequently 1000 μL aliquots of diluted bacteria suspensions were supplemented with 2 PU catheter's pieces (0.5 cm length) coated with nanogel loaded either with triafluocyl (0.5 mg/mL) or triafluocyl-TPGS (0.5 mg/mL w/w). Non coated PU catheter served as control. Nanogel coating was prepared according to the aforementioned protocol described in examples 1 and 4. Bacterial solutions with catheters were incubated for 30 min at 37° C. and 220 rpm. Subsequently catheters were washed 2 times with saline (0.9% NaCl) and placed into the dedicated non-activated inserts of a 48-well plate with 300 μL of fresh TSB medium and grown for 24 h under static conditions at 37° C. using Calscreener.

In FIG. 7, the real-time microcalorimetric measurements of bacterial metabolic activity upon adhesion to catheters, is expressed as the heat flow. Solid line—control non-coated PU catheter; dashed line—PU catheter coated with triafluocyl-loaded nanogel; dotted line—PU catheter coated with triafluocyl-TPGS-loaded nanogel.

Nanogel coating with triafluocyl can increase anti-adhesive property of PU catheter which is seen as the delay of the peak metabolic rate. Presence of TPGS in the coating can further shift the peak of bacterial growth depicting less bacterial attachment on the catheter surface.

### Example 8 Combination of Triafluocyl-TPGS with Antimicrobial Agent Minocycline Confers the Nanogel (NTTM) Coating a Long-Term Anti-Adhesion Property Against S. aureus

S. aureus (ATCC BAA-1556, MRSA) was grown overnight in TSB (tryptic soy broth) medium, before being diluted 1×10⁶ in fresh TSB. Subsequently 1000 μL aliquots of diluted bacteria suspensions were supplemented with 2 catheter segments (0.5 cm length) coated with nanogel loaded either with triafluocyl (0.05 mg/mL, NTM) or triafluocyl-TPGS (0.1/0.2 mg/mL w/w, NTTM) supplemented with minocycline as a bacteriostatic agent.

Preparation of the Nanogel Solution with Pox(mDOPA) and PAH (NG)

Aqueous solution of PAH (0.5 mL, 0.5 mg/ml) at pH 10 was slowly added under stirring (300 rpm) with a magnetic stirrer, to Pox(rDOPA) (5 ml, 0.5 mg/ml). The solution was allowed to react for one hour at room temperature under vigorous stirring (500 rpm).

Nanogels (NG) with a diameter ranging from 100 nm to 300 nm were observed in water suspension by Dynamic Light Scattering (Zetasizer Advance Pro, Malvern).

Nanogel coating of the catheters was performed according to the aforementioned protocol described in example 3. Minocycline (0.5 mg/mL) was added to the last layer of the nanogel, either to NG or NT or NTT resulting respectively in the formation of the following variants, NGM, NTM or NTTM. Non-coated catheter segments served as control. Two sets of coated catheter segments were tested, namely “washed” after 20 days of incubation in PBS/DMSO 2% (long-term drug release assay) and “non-washed” (not incubated in the buffer, time=0 days) in order to observe the length of anti-adhesion activity of the tested coatings.

For testing the anti-adhesion property of the nanogel coating, aforementioned bacterial solutions with catheters were incubated for 30 min at 37° C. and 220 rpm. Subsequently catheters were washed 2 times with saline (0.9% NaCl) and placed into the dedicated non-activated inserts of a 48-well plate with 300 μL of fresh TSB medium and grown for 24 h under static conditions at 37° C. using Calscreener. The microcalorimetric read-out was acquired.

In FIG. 8, the real-time microcalorimetric measurements of bacterial metabolic activity upon adhesion to catheters, expressed as the heat flow. A—Baseline growth of S. aureus (MRSA) at the dilution of 10⁻⁶ (no catheter segments); B—control non-coated catheter segment; C—NGM—catheter segment coated with the 5 layers of nanogel loaded with minocycline 0.5 mg/mL added in the last nanogel layer and grafted with PEG (t=0 days); D—NTM—catheter segment coated with the 5 layers of nanogel loaded with triafluocyl 0.05 mg/mL and followed by minocycline (0.5 mg/mL) added to the last layer of nanogel and grafted with PEG (t=0 days); E—NTTM—catheter segment coated with the 5 layers of nanogel loaded with the combination of Triafluocyl/TPGS (0.1/0.2 mg/mL, w/w) and followed by minocycline (0.5 mg/mL) added to the last layer of nanogel (t=0 days); F—NGM—catheter segment coated with the 5 layers of nanogel loaded with minocycline 0.5 mg/mL added in the last nanogel layer and grafted with PEG (t=20 days); G—NTM—catheter segment coated with the 5 layers of nanogel loaded with triafluocyl 0.05 mg/mL followed by minocycline (0.5 mg/mL) added to the last layer of nanogel and grafted with PEG (t=20 days); H—NTTM—catheter segment coated with the 5 layers of nanogel loaded with the combination of Triafluocyl/TPGS (0.1/0.2 mg/mL, w/w) followed by minocycline (0.5 mg/mL) added to the last layer of nanogel (t=20 days).

Introducing minocycline to the coating results in a full reduction of bacterial adhesion to the catheters NGM, NTM and NTTM as seen as a background level at the beginning of the drug releasing test (at 0-day time, plots C, D, E).

Anti-attachment effect of coated catheters, particularly NTM and NTTM (plots G and H) can last for at least 20 days (duration of the release of the drugs in this experimental setting). Compared to the non-triafluocyl loaded coating, NGM, there is a clear synergic effect for triafluocyl and minocycline (NTM, NTTM). This effect is potentiated in the presence of TPGS (NTTM) seen as a shift and reduction of the microcalorimetric signal.

Deploying the combination of triafluocyl with TPGS gives a possibility to increase the amounts of loaded triafluocyl which results in a stronger synergy between triafluocyl and minocycline (NTTM vs. NTM). This clearly shows a superiority of the coating NTTM over NTM indicated by more pronounced and sustained anti-adhesion effect in the long-term perspective (>20 days).

### Example 9: Preparation of the Nanogel According to the Invention with P(mDOPA), Chlorhexidine Chloride and TPGS (Nanogel NCcT)

The nanogel preparation is similar to the one disclosed in the example 1, except for the drug loading we use Chlorhexidine chloride as Active Pharmaceutical Ingredient and TPGS as amphiphilic molecule.

### 9.1 Oxidation of PmDOPA→Pox(mDOPA).

A homopolymer of methacrylamide bearing 3,4-dihydroxy-L-phenylalanine (P(mDOPA), is oxidized in aqueous media under basic conditions for 12 hours to form water-soluble Pox(mDOPA). Oxidized catechol moieties of Pox(mDOPA) are necessary for the covalent interaction of PAH through amine/quinone reaction and/or Schiff base formation at room temperature, and consequently for the preparation of stable cross-linked nanogel.

P(mDOPA) (2.5 mg) was dissolved in distilled water (5 mL) and NaOH (0.1 M) was slowly added in order to raise the pH above 10 and to promote the oxidation of catechol groups of P(mDOPA).

### 9.2 Preparation of Chlorhexidine Chloride/TPGS Mixture in DMSO

Chlorhexidine chloride (Chc) and TPGS were dissolved separately in DMSO to prepare stock solutions of 10 mg/mL of Chc and TPGS. 115 μL of Chc solution and 234 μl of TPGS solution were mixed under stirring (300 rpm) during 10 min at RT.

9.3 Preparation of the Nanogel with Chlorhexidine Chloride/TPGS and PAH

Pox(mDOPA) (5 ml, 0.5 mg/ml) was added under stirring (300 rpm) to the mixture obtained at point x.2. After one hour of homogenization at RT, an aqueous solution of PAH (0.5 mL, 0.5 mg/ml) at pH 10 was slowly added to the mixture solution. The solution was allowed to react for one hour at room temperature under vigorous stirring (500 rpm).

Nanogels (NCcT) with a diameter ranging from 100 nm to 350 nm were observed in liquid suspension by Dynamic Light Scattering (Zetasizer Advance Pro, Malvern).

Comparison of the Nanogel NCcT According to the Invention to the Nanogel NCc without Using TPGS: Particle Size and Stability

The nanogel particle diameter size according to the example 9 of the invention is compared to the one obtained without TPGS (NCc).

Nanogel Preparation with Chlorhexidine Chloride (NCc):

P(mDOPA) (2.5 mg) was dissolved in distilled water (5 mL) and NaOH (0.1 M) was slowly added in order to raise the pH above 10 and to promote the oxidation of catechol groups of P(mDOPA). After one night at room temperature, 0.110 ml of Chc solution (10 mg/ml in DMSO) was added dropwise to the solution of Pox(mDOPA) under stirring (300 rpm) at room temperature. After one hour of homogenization, an aqueous solution of PAH (0.5 mL; 0.5 g/L) at pH 10 was slowly added to the mixture solution. The solution was allowed to react for one hour at room temperature under vigorous stirring (500 rpm). Nanogels (NC) with a large diameter higher than 500 nm were observed in the mixture solution by Dynamic Light Scattering (Zetasizer Advance Pro, Malvern).

Loaded nanogel with Chc in the presence of TPGS (NCcT) present a lower diameter ranging from 100 nm to 350 nm with a lower size distribution compared to the nanogel diameter without TPGS (NCc) as illustrated in FIG. 9.

### Example 10: Preparation of the Nanogel According to the Invention with P(mDOPA), Fluometacyl®, Chlorhexidine Chloride and TPGS (Nanogel NFCcT)

The nanogel preparation is similar to the one disclosed in the example 1, except for the drug loading, we use Fluometacyl® (Fluo) and Chlorhexidine chloride and TPGS as amphiphilic molecule.

### 10.1 Oxidation of PmDOPA→Pox(mDOPA).

A homopolymer of methacrylamide bearing 3,4-dihydroxy-L-phenylalanine (P(mDOPA), is oxidized in aqueous media under basic conditions for 12 hours to form water-soluble Pox(mDOPA). Oxidized catechol moieties of Pox(mDOPA) are necessary for the covalent interaction of PAH through amine/quinone reaction and/or Schiff base formation at room temperature, and consequently for the preparation of stable cross-linked nanogel in aqueous media.

P(mDOPA) (2.5 mg) was dissolved in distilled water (5 mL) and NaOH (0.1 M) was slowly added in order to raise the pH above 10 and to promote the oxidation of catechol groups of P(mDOPA).

### 10.2 Preparation of Fluometacyl*, Chlorhexidine Chloride/TPGS Mixture in DMSO

Chlorhexidine chloride (Chc) and TPGS were dissolved separately in DMSO to prepare stock solutions of 10 mg/mL of Fluo, Chc and TPGS. 115 μL of Fluometacyl® solution, 115 μL of Chc solution and 234 μl of TPGS solution were mixed under stirring (300 rpm) during 10 min at RT.

10.3 Preparation of the Nanogel with Fluo, Chc/TPGS and PAH

Pox(mDOPA) (5 ml, 0.5 mg/ml) was added under stirring (300 rpm) to the mixture solution obtained at point 12.2. After one hour of homogenization at RT, an aqueous solution of PAH (0.5 mL, 0.5 mg/ml) at pH 10 was slowly added to the mixture solution. The solution was allowed to react for one hour at room temperature under vigorous stirring (500 rpm).

Nanogels (NFCcT) with a diameter ranging from 100 nm to 350 nm were observed in the mixture solution by Dynamic Light Scattering (Zetasizer Advance Pro, Malvern).

Comparison of the Nanogel NFCcT According to the Invention to the Nanogel NFCc without TPGS.

The nanogel diameter size according to the example 10 of the invention is compared to the nanogel loaded with Fluo and Chc and without TPGS (NFCc).

Nanogel Preparation with Chlorhexidine Chloride (NFCc):

P(mDOPA) (2.5 mg) was dissolved in distilled water (5 mL) and NaOH (0.1 M) was slowly added in order to raise the pH above 10 and to promote the oxidation of catechol groups of P(mDOPA). After one night at room temperature, we added respectively 0.11 ml of Chc solution (10 mg/ml in DMSO) flowed by 0.11 ml of Fluo solution (10 mg/ml in DMSO) to the solution of Pox(mDOPA) under stirring (300 rpm) at room temperature. After one hour of homogenization, an aqueous solution of PAH (0.5 mL; 0.5 g/L) at pH 10 was slowly added to the mixture solution. The solution was allowed to react for one hour at room temperature under vigorous stirring (500 rpm). Nanogel (NFCc) with a large diameter higher than 500 nm is observed in the mixture solution by Dynamic Light Scattering (Zetasizer Advance Pro, Malvern).

Loaded nanogel with Fluometacyl® and Chc in the presence of TPGS (NFCcT) present a lower particle diameter ranging from 100 nm to 350 nm with a lower size distribution compared to the nanogel diameter without TPGS (NFCc) as illustrated in FIG. 10.

After 24 h, increased size of NFCc was observed, depicting nanogel aggregate formation and precipitation, which was not observed for NFCcT that remained stable after 24 h. (FIG. 11)

### Example 11: Anti-Attachment Property of Nanogel-Coated Catheters Loaded with Fluometacyl® and Chlorhexidine in the Presence of TPGS or not, Tested for Pseudomonas aeruginosa

### 11.1 Preparation of the Test Samples for the Microcalorimetric Measurements

P. aeruginosa (ATCC 15442) was grown overnight in TSB (tryptic soy broth) medium, before being diluted 1×10⁷ fold in the fresh TSB. Subsequently 1000 mL aliquots of diluted bacteria suspensions were supplemented with 2 PU catheter segments (0.5 cm length) coated with the 5 layers of nanogel loaded with Fluometacyl® (0.2 mg/mL) and chlorhexidine chloride (0.2 mg/mL) either with TPGS (0.4 mg/mL) or not. Nanogel coating was prepared according to the protocol below to which respective bioactive molecules were added.

Bacterial solutions with catheters were incubated for 30 min at 37 C and 220 rpm. Subsequently catheters were washed 2 times with saline (0.9% NaCl) and placed into the dedicated non-activated inserts of a 48-well plate with 300 mL of fresh TSB medium and grown for 24 h under static conditions at 37 C using Calscreener.

11.2 Preparation of the Nanogel (NFCc and NFCcT) with Pox(mDOPA) and PAH and Drug Loading (Fluometacyl® and Chlorhexidine)

Aqueous solution of PAH (0.5 mL, 0.5 mg/ml) at pH 10 was slowly added under stirring (300 rpm) with a magnetic stirrer, to Pox(mDOPA) (5 ml, 0.5 mg/ml) that was previously preincubated with Fluometacyl® (0.2 mg/mL) and chlorhexidine chloride (CHLXc, 0.2 mg/mL) with or without TPGS (0.4 mg/mL) for 10 minutes. The solution was allowed to react for one hour at room temperature under vigorous stirring (500 rpm).

Nanogels with a diameter ranging from 100 nm to 300 nm were observed in water suspension by Dynamic Light Scattering (Zetasizer Advance Pro, Malvern).

Test drugs were kept frozen as the following stock solutions, Fluometacyl® (3.3 mg/mL stock in DMSO, prepared according to Eur J Med Chem 2020 Dec. 15:208:112767, chlorhexidine chloride (3.3 mg/mL stock in DMSO, Merck), TPGS (3.3 mg/mL stock in DMSO, Merck).

FIG. 12 shows the real-time microcalorimetric measurements of bacterial metabolic activity upon adhesion to catheters, expressed as the heat flow. Curve A—control bacterial growth for P. aeruginosa 10⁻⁷; Curve B—Bacteria with the nanogel-coated PU catheter loaded with Fluometacyl® and chlorhexidine chloride (NFCc); Curve C—Bacteria with the nanogel-coated PU catheter loaded with Fluometacyl® and chlorhexidine chloride in the presence of TPGS (NFCcT).

Nanogel coating with Fluometacyl® and chlorhexidine chloride in the presence of TPGS confers the higher anti-adhesive property of PU catheter compared to the coating that is lacking TPGS. This is seen as the shift in the time to peak of the metabolic activity of the bacteria grown from the population that adhered to the catheters during the initial incubation. In other words, less bacteria are attached to the catheter segments and the metabolic activity appears later.

TPGS as the amphiphilic molecule enhances the entrapment of antibacterial molecules of hydrophobic and hydrophilic nature such as Fluometacyl® and chlorhexidine into the nanogel coating.

### Example 12: Nanogel-Coated Catheters Loaded with Fluometacyl® and Chlorhexidine Display Stronger Anti-Adhesion Property Against Pseudomonas aeruginosa in the Presence of TPGS

### 12.1 Preparation of the Test Samples for the Microcalorimetric Measurements

P. aeruginosa (ATCC 15442) was grown overnight in TSB (tryptic soy broth) medium, before being diluted 1×10⁷ fold in the fresh TSB. Subsequently 1000 mL aliquots of diluted bacteria suspensions were supplemented with 2 PU catheter segments (0.5 cm length) coated with the 5 layers of nanogel loaded with Fluometacyl® (0.2 mg/mL) and soaked (1 hr) in the aqueous solution of chlorhexidine chloride (1 mg/mL) or with 2 PU catheter segments (0.5 cm length) coated with the 5 layers of nanogel loaded with Fluometacyl® (0.2 mg/mL) and chlorhexidine chloride (0.2 mg/mL) in the presence of TPGS (0.4 mg/mL) or without. Nanogel coating was prepared according to the protocol below to which respective bioactive molecules were added.

Bacterial solutions with catheters were incubated for 30 min at 37 C and 220 rpm. Subsequently catheters were washed 2 times with saline (0.9% NaCl) and placed into the dedicated non-activated inserts of a 48-well plate with 300 mL of fresh TSB medium and grown for 24 h under static conditions at 37 C using Calscreener.

12.2 Preparation of the Nanogel (NFCcT) with Pox(rDOPA) and PAH and Drug Loading

Aqueous solution of PAH (0.5 mL, 0.5 mg/ml) at pH 10 was slowly added under stirring (300 rpm) with a magnetic stirrer, to Pox(mDOPA) (5 ml, 0.5 mg/ml) that was previously preincubated with Fluometacyl® (0.2 mg/mL) and chlorhexidine chloride (Cc, 0.2 mg/mL) with or without TPGS (0.4 mg/mL) for 10 minutes. The solution was allowed to react for one hour at room temperature under vigorous stirring (500 rpm). Alternatively, instead of loading the nanogel with both Fluometacyl® and chlorhexidine chloride, the nanogel-coated catheters with Fluometacyl® were soaked in the aqueous solution of Chc (1 mg/mL) for 1 h under agitation (orbital shaker) in order to load chlorhexidine separately from Fluometacyl®.

Nanogels with a diameter ranging from 100 nm to 300 nm were observed in water suspension by Dynamic Light Scattering (Zetasizer Advance Pro, Malvern).

Test drugs were kept frozen as the following stock solutions, Fluometacyl® (3.3 mg/mL stock in DMSO, prepared according to Eur J Med Chem 2020 Dec. 15:208:112767), chlorhexidine chloride (3.3 mg/mL stock in DMSO, Merck), TPGS (3.3 mg/mL stock in DMSO, Merck). For the soaking protocol fresh aqueous solution of 1 mg/mL was prepared from the powder.

FIG. 13 shows the real-time microcalorimetric measurements of bacterial metabolic activity upon adhesion to catheters, expressed as the heat flow. Curve A—Bacteria with the nanogel-coated PU catheter loaded with Fluometacyl® and chlorhexidine chloride (NFCc); Curve B—Bacteria with the nanogel-coated PU catheter loaded with Fluometacyl® and soaked in the solution of chlorhexidine chloride (1 mg/mL) (NF/Cc); Curve C—Bacteria with the nanogel-coated PU catheter loaded with Fluometacyl® and chlorhexidine chloride in the presence of TPGS (NFCcT).

In the presence of TPGS the nanogel coating carrying Fluometacyl® and chlorhexidine confers the strongest anti-attachment properties seen as a forward-shifted peak metabolic signal. Soaking in chlorhexidine gives a moderate property compared to the approach with the concomitant loading of Fluometacyl® and chlorhexidine. This supports a positive effect of TPGS for loading bioactive molecules in the nanogel coating.

### Example 13 Stabilization of the Mixture Fluometacyl®, Chlorhexidine, Polymixin B with and without TPGS

The nanogel preparation is similar to the one disclosed above for NF and NFT excepted that we use a mixture of Fluometacyl®, Chlorhexidine and Polymyxin B keeping the same concentration (0.2 mg/ml).

Stabilization of different Active Pharmaceutical Ingredients or drugs may be problematic due to interactions that can be exist between molecules. In this example we show clearly the added value of the presence of TPGS to enhance stability of the nanogel in liquid suspension. Nanogel NFCcP with the three different drugs or APIs without TPGS generates a big aggregate with very low stability. But in the presence of TPGS, NFCcPT nanogel shows a better stability and DLS analysis show nanogel diameter around micron size in the liquid suspension. In FIG. 14, the mixture of nanogels NFT, NCcT and NPT shows nanogel diameter ranging from 100 nm to 350 nm with higher stability in the liquid suspension.

### Example 14 Preparation of the Nanogel (NFCy)) According to the Invention with P(mDOPA) Fluometacyl® and Hydroxypropyl-Beta-Cyclodextrin

The nanogel preparation is similar to the one disclosed above excepted we use hydroxypropyl-beta-cyclodextrin (also called cyclodextrin hereafter) instead of TPGS.

### 14.1 Oxidation of PmDOPA→Pox(mDOPA).

A homopolymer of methacrylamide bearing 3,4-dihydroxy-L-phenylalanine (P(mDOPA) is oxidized in aqueous media under basic conditions for 12 hours to form hydrosoluble Pox(mDOPA). Oxidized catechol moieties of Pox(mDOPA) are necessary for the covalent interaction of PAH through amine/quinone reaction and/or Schiff base formation at room temperature, and consequently for the preparation of stable cross-linked nanogel in liquid dispersion.

P(mDOPA) (2.5 mg) was dissolved in distilled water (5 mL) and NaOH (0.1 M) was slowly added in order to raise the pH above 10 and to promote the oxidation of catechol groups of P(mDOPA).

### 14.2 Preparation of Fluometacyl®/Hydroxypropyl-Beta-Cyclodextrin Mixture in DMSO

75 μl of hydroxypropyl-beta-cyclodextrin solution provided by Merck 40%(400 mg/ml in H2O) and 60 μl of Fluometacyl solution (3.3 mg/ml in DMSO) were mixed under stirring (300 rpm) with a magnetic stirrer.

14.3 Preparation of the Nanogel with Fluometacyl®/Hydroxypropyl-Beta-Cyclodextrin and PAH

Pox(mDOPA) (0.87 ml, 0.5 mg/ml) was added under stirring (300 rpm) with a magnetic stirrer, to the mixture obtained at point 14.2. DLS analysis FIG. 15 shows the presence of species lower then 10 nm which characterize cyclodextrin molecules. After one hour of homogenization at RT, an aqueous solution of PAH (0.087 mL, 0.5 mg/ml) at pH 10 was slowly added to the mixture solution. The solution was allowed to react for one hour at room temperature under vigorous stirring (500 rpm) with a magnetic stirrer. FIG. 15 shows Nanogels NFCy (dashed line) with a diameter ranging from 100 nm to 350 nm in liquid suspension compared to solution before addition of PAH (absence of nanogel) (solid line) as observed by Dynamic Light Scattering (Zetasizer Advance Pro, Malvern).

### Example 15: Stability Comparison of the Nanogels NF, NFT and NFCy According to the Invention with Different Concentration of Fluometacyl® and TPGS or Hydroxypropyl-Beta-Cyclodextrin as Amphiphilic Molecule

The nanogel preparation is similar to the one disclosed above for NF, NFT and NFCy excepted we use different concentration of Fluometacyl® (0.2, 0.4 and 0.8 mg/ml) and we keep the same ration with the amphiphilic molecule (Fluo:TPGS 1:2 and Fluo:cyclodextrin 1:75).

Size and polydispersity of the different nanogels are followed by DLS at day 0, 1 and 4. We can see clearly that with enhancing the concentration of Fluometacyl the size and polydispersity of nanogels NF increase inducing formation of precipitation. However, in the presence of amphiphilic molecule TPGS and hydroxypropyl-beta-cyclodextrin consider as stabilizers, nanogels NFT and NFCy present a higher stability.

### Example 16 Hydroxypropyl-β-Cyclodextrin in Contrast to TPGS Doesn't Impact Antibacterial Effect of Fluometacyl® in Liquid Medium, Tested Against Staphylococcus aureus

### 16.1 Preparation of the Test Samples for the Microcalorimetric Measurements

S. aureus (MRSA, ATCC 6538) was grown overnight in TSB (tryptic soy broth) medium, before being diluted 1×10⁷ fold in the fresh TSB. Subsequently 1000 mL aliquots of diluted bacteria suspensions were supplemented with Fluometacyl® (10 or 20 μg/mL) or mixture of Fluometacyl® (10 or 20 μg/mL) and hydroxypropyl-β-cyclodextrin (42 μg/mL). Subsequently 300 μL of suspensions were placed into the dedicated non-activated inserts of a 48-well plate and allowed for growing for 24 h under static conditions at 37 C using Calscreener.

### 16.2 Preparation of Stock Solutions

Fluometacyl® (prepared according to Eur J Med Chem 2020 Dec. 15:208:112767) was kept frozen as the following stock solution of 3.3 mg/mL in EtOH. Hydroxypropyl-β-cyclodextrin (Merck) was kept as powder at RT and the appropriate stock solution in H₂O was prepared freshly prior to use.

FIG. 17 shows the real-time microcalorimetric measurements of bacterial metabolic activity expressed as the heat flow. Curve A—control bacterial growth for S. aureus MRSA 10⁻⁶; Curve B—Bacteria with Fluometacyl® (10 μg/mL); Curve C—Bacteria with Fluometacyl® (10 μg/mL) and hydroxypropyl-β-cyclodextrin (42 μg/mL); Curve D—Bacteria with Fluometacyl® (20 μg/mL); Curve E—Bacteria with Fluometacyl® (20 μg/mL) and hydroxypropyl-β-cyclodextrin (42 μg/mL).

In solution, hydroxypropyl-β-cyclodextrin compared to TPGS (in previous examples) didn't impair the antibacterial activity of Fluometacyl®.

### Example 17: Anti-Attachment Property of Nanogel-Coated Catheters Loaded with Fluometacyl® in the Presence of TPGS or Hydroxypropyl-β-Cyclodextrin, Tested Against Staphylococcus aureus

### 17.1 Preparation of the Test Samples for the Microcalorimetric Measurements

S. aureus (MRSA, ATCC 6538) was grown overnight in TSB (tryptic soy broth) medium, before being diluted 1×10⁷ fold in the fresh TSB. Subsequently 1000 mL aliquots of diluted bacteria suspensions were supplemented with 2 PU catheter segments (0.5 cm length) coated with the 5 layers of nanogel loaded with Fluometacyl® (0.2 mg/mL) and TPGS (0.4 mg/mL) or Fluometacyl® (0.2 mg/mL) and hydroxypropyl-β-cyclodextrin (15 mg/mL). Nanogel coating was prepared according to the protocol below to which respective bioactive molecules were added.

Bacterial suspensions were incubated with catheters for 30 min at 37 C and 220 rpm. Subsequently catheters were washed 2 times with saline (0.9% NaCl) and placed into the dedicated non-activated inserts of a 48-well plate with 300 mL of fresh TSB medium and grown for 24 h under static conditions at 37 C using Calscreener.

17.2 Preparation of the Nanogel with Pox(mDOPA) and PAH (NG) and Drug Loading

Aqueous solution of PAH (0.5 mL, 0.5 mg/ml) at pH 10 was slowly added under stirring (300 rpm) with a magnetic stirrer, to Pox(mDOPA) (5 ml, 0.5 mg/ml) that was previously preincubated either with Fluometacyl® (0.2 mg/mL) and TPGS (0.4 mg/mL) or Fluometacyl® (0.2 mg/mL) and hydroxypropyl-β-cyclodextrin (15 mg/mL) for 10 minutes. The solution was allowed to react for one hour at room temperature under vigorous stirring (500 rpm).

Nanogels with a diameter ranging from 100 nm to 300 nm were observed by Dynamic Light Scattering (Zetasizer Advance Pro, Malvern).

Test drugs were kept frozen as the following stock solutions, Fluometacyl® (3.3 mg/mL stock in DMSO, prepared according to Eur J Med Chem 2020 Dec. 15:208:112767), TPGS (3.3 mg/mL stock in DMSO, Merck). Hydroxypropyl-β-cyclodextrin (Merck) was kept as powder at RT and the appropriate stock solution in H₂O was prepared freshly prior to use.

FIG. 18 shows the real-time microcalorimetric measurements of bacterial metabolic activity upon adhesion to catheters, expressed as the heat flow. Curve A—control bacterial growth for S. aureus MRSA 10⁻⁶; Curve B—Bacteria with the nanogel-coated PU catheter loaded with Fluometacyl® and TPGS (0.2/0.4, w/w); Curve C—Bacteria with the nanogel-coated PU catheter loaded with Fluometacyl® and hydroxypropyl-β-cyclodextrin (0.2/15, w/w).

Nanogel coating with Fluometacyl® and hydroxypropyl-β-cyclodextrin confers the higher anti-adhesion property of the PU catheter compared to the coating with TPGS. This is seen as the reduction of the signal and the shift in the time to peak of the metabolic activity of the bacteria grown from the population that adhered to the catheters during the initial incubation. In other words, less bacteria attached to the catheter segments, later the metabolic activity appears.

Hydroxypropyl-β-cyclodextrin enhances the entrapment of Fluometacyl® into the nanogel coating more than TPGS.

1. A nanogel made of a poly(methacrylamide)-bearing quinone groups of formula (1) 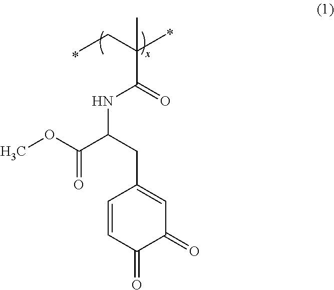
wherein x is an integer >1, preferably x is between 1 and 100
and/or
made of a poly(vinylquinone) represented by formula (6) 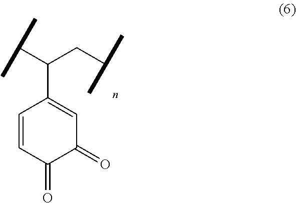
wherein n is an integer >1, preferably n is between 1 and 100;
and crosslinked with a polymer bearing primary or secondary amine groups;
wherein the nanogel comprises one or more bioactive molecule, therapeutic molecule or drug together with an amphiphilic molecule.

2. The nanogel according to claim 1 wherein the amphiphilic molecule is a vitamin-E derivative, preferably D-α-tocophenyl polyethylene glycol succinate.

3. The nanogel according to claim 1 wherein the amphiphilic molecule is an amphiphilic cyclodextrin, preferably hydroxypropyl-beta-cyclodextrin.

4. The nanogel according to any one of claims 1 to 3 wherein the polymer bearing primary or secondary amine groups is poly-(allylamine hydrochloride) of formula (2) 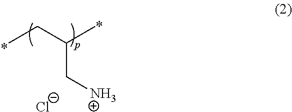
wherein p is an integer >1; preferably p is between 10 and 300.

5. The nanogel according to any one of claims 1 to 4 wherein the bioactive molecule, therapeutic molecule or drug is a triazolo(4,4-d)-pyrimidine derivative of formula (3) 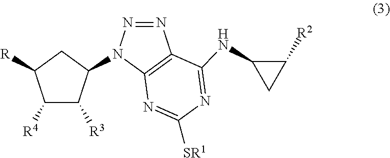
wherein R1 is C3-5 alkyl optionally substituted by one or more halogen atoms;
R₂ is a phenyl group, optionally substituted by one or more halogen atoms; R₃ and R₄ are both hydroxyl; R is OH or XOH, wherein X is CH₂, OCH₂CH₂, or a bond;
or a pharmaceutical acceptable salt or solvate thereof, or a solvate thereof or a solvate of such a salt provided that when X is CH₂ or a bond, R₁ is not propyl;
when X is CH₂ and R₁ CH₂CH₂CF₃, butyl or pentyl, the phenyl group at R₂ must be substituted by fluorine; when X is OCH₂CH₂ and R₁ is propyl, the phenyl group at R₂ must be substituted by fluorine;
preferably the triazolo (4,4-d)-pyrimidine derivative is (1S,2S,3R,5S)-3-[7-[(1R,2S)-2-(3,4-difluorophenyl)cyclopropylamino]-5-(propylthio)-3H-[1,2,3]-triazolo[4,5-d]pyrimidin-3-yl]-5-(2-hydroxyethoxy)-1,2-cyclopentanediol) also called Triafluocyl; or is (1S,2R,3S,4R)-4-[7-[[(1R,2S)-2-(3,4-Difluorophenyl)-cyclopropyl]amino]-5-(propylthio)-3H-1,2,3-triazolo[4,5-d]pyrimidin-3-yl]-1,2,3-cyclopentanetriol also called Fluometacyl or Fluometacyl® or is a combination thereof

6. The nanogel according to any one of claims 1 to 4 wherein the bioactive molecule, therapeutic molecule or drug is a pyrimidine derivative represented by formula (4) 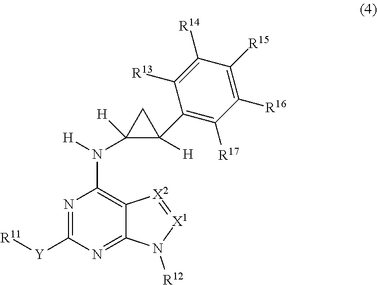
or optical isomers, racemic mixtures thereof, pharmaceutically acceptable acid addition salts, pharmaceutically acceptable metal salts, or alkylated ammonium salts or prodrug thereof;
wherein:
X¹ and X² are independently N, CH, CR⁸ wherein R⁸ is C₁₋₆ alkyl, C₂₋₆ alkenyl or C₂₋₆ alkynyl; with the exception that if one of X¹ or X² is equal to N, then the remaining X¹ or X² are selected from CH, CR⁸.
—Y— is —O— or —S—;
R¹¹ and R¹² are independently C₁₋₆-alkyl, C₂₋₆-alkenyl, C₂₋₆-alkynyl, C₃₋₆-cycloalkyl, aryl, aryl-C₁₋₆-alkyl wherein the alkyl or cycloalkyl moiety is optionally mono or polysubstituted with OH or an halogen and the aryl moiety is optionally mono or polysubstituted with an halogen, —C₁₋₆alkyl, —C₁₋₆alkoxy, —OH, —NO₂, —CN, —NH₂, —NHR⁸, —N(R⁸)₂—COOH, —COOR⁸, —CONH₂, —CONHR⁸, —CON(R⁸)₂, —SO₂NH₂, —SO₂NHR⁸, or —SO₂N(R⁸)₂;
R¹³, R¹⁴, R¹⁵, R¹⁶ and R¹⁷ are independently H, an halogen, a C₁₋₆alkyl, C₁₋₆alkoxy, —OH, —NO₂, —CN, —NH₂, —NHR⁸, —N(R⁸)₂—COOH, —COOR⁸, —CONH₂, —CONHR⁸, —CON(R⁸)₂, —SO₂NH₂, —SO₂NHR⁸, or —SO₂N(R⁸)₂.

7. The nanogel according to claim 2 wherein the bioactive molecule, therapeutic molecule or drug and D-α-tocophenyl polyethylene glycol succinate are in a ratio between 1:1 w/w to 1:5 w/w.

8. The nanogel according to claim 3 wherein the bioactive molecule, therapeutic molecule or drug and hydroxypropyl-beta cyclodextrin are in a ratio between 1:1 w/w to 1:200 w/w.

9. The nanogel according to any one of claims 1 to 8 wherein the one or more bioactive molecule, therapeutic molecule or drug are an antivirulence or a bactericidal agent together with a bacteriostatic agent, preferably minocycline or chlorhexidine.

10. The nanogel according to any one of claims 1 to 9, wherein the bioactive molecule, therapeutic molecule or drug are a combination of Fluometacyl or Fluometacyl® with chlorhexidine.

11. The nanogel according to any one of claims 1 to 10, wherein the bioactive molecule, therapeutic molecule or drug are a combination of Fluometacyl or Fluometacyl® with chlorhexidine and PolymixinB.

12. The nanogel according to any one of claims 1 to 11 for use in treatment or prevention of bacterial infection.

13. The nanogel according to any one of claims 1 to 12 for use in topical administration.

14. A biomaterial implant, medical device or bioprosthesis wherein a surface or part thereof is coated with the nanogel according to any one of claims 1 to 11.

15. The biomaterial implant, medical device according to claim 14 wherein the medical device is a catheter.

16. A method of making a nanogel comprising one or more bioactive molecule, therapeutic molecule or drug, together with an amphiphilic molecule, wherein the nanogel is obtained by one of both following methods, depending whether each bioactive molecule, therapeutic molecule or drug is to be loaded separately or simultaneously with the amphiphilic molecule in the nanogel:
a) when each bioactive molecule, therapeutic molecule or drug is loaded separately with the amphiphilic molecule,
the method comprises the following in-sequence steps:
i) mixing poly(methacrylamide)-bearing quinone groups of formula (1) 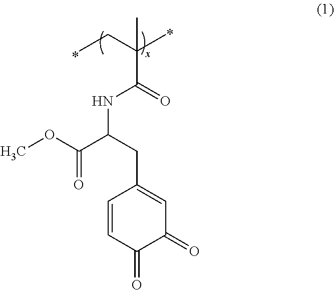
wherein x is an integer >1, preferably x is between 1 and 100
and/or a poly(vinylquinone) represented by formula (6), 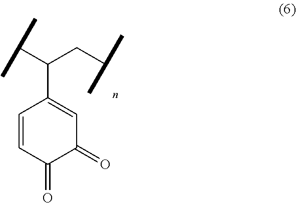
wherein n is an integer >1, preferably n is between 1 and 100;
with only one bioactive molecule, therapeutic molecule or drug together with an amphiphilic molecule;
ii) adding a solution of polymer bearing primary or secondary amine groups to the resulting mixture obtained in step i) to generate a crosslinked nanogel comprising one bioactive molecule, therapeutic molecule or drug together with an amphiphilic molecule;
iii) repeating step 1) and ii) for each additional bioactive molecule, therapeutic molecule or drug;
iv) mixing each crosslinked nanogel obtained at step iii) to obtain a resulting nanogel comprising two or more biological molecules, therapeutic molecules or drugs together with an amphiphilic molecule.
b) when the bioactive molecules, therapeutic molecules or drugs are loaded simultaneously with the amphiphilic molecule; the method comprises the following in-sequence steps:
i) mixing poly(methacrylamide)-bearing quinone groups of formula (1) 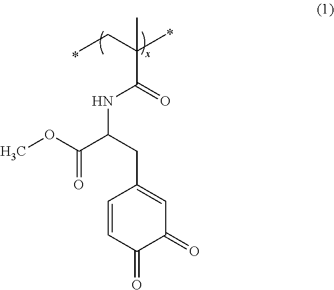
wherein x is an integer >1, preferably x is between 1 and 100
and/or a poly(vinylquinone) represented by formula (6) 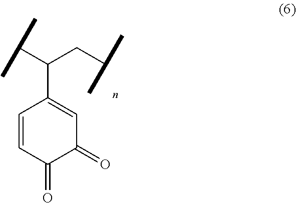
wherein n is an integer >1, preferably n is between 1 and 100;
with a one or more bioactive molecule, therapeutic molecule or drug together with an amphiphilic molecule;
ii) adding a solution of polymer bearing primary or secondary amine groups to the resulting mixture of step i) to generate a nanogel comprising one or more bioactive molecule, therapeutic molecule or drug together with the amphiphilic molecule.

17. The method according to claim 16, wherein the amphiphilic molecule is a vitamin-E derivative, preferably D-α-tocophenyl polyethylene glycol succinate.

18. The method according to claim 16, wherein the amphiphilic molecule is an amphiphilic cyclodextrin, preferably hydroxypropyl-beta-cyclodextrin.

19. The method according to claim 16 wherein the ratio of bioactive molecule, therapeutic molecule or drug with D-α-tocophenyl polyethylene glycol succinate is between 1:1 w/w and 1:5 w/w.

20. The method according to claim 16 wherein the ratio of bioactive molecule, therapeutic molecule or drug with hydroxypropyl-beta-cyclodextrin is between 1:1 and 1:200.

21. The method according to any one of claims 16 to 20 wherein the one or more bioactive molecule, therapeutic molecule or drug is a triazolo(4,4-d)-pyrimidine derivative of formula (3); preferably the triazolo(4,4-d)-pyrimidine derivative is (1S,2S,3R,5S)-3-[7-[(1R,2S)-2-(3,4-difluorophenyl)cyclopropylamino]-5-(propylthio)-3H-[1,2,3]-triazolo[4,5-d]pyrimidin-3-yl]-5-(2-hydroxyethoxy)-1,2-cyclopentanediol) also called Triafluocyl; or (1S,2R,3S,4R)-4-[7-[[(1R,2S)-2-(3,4-Difluorophenyl)-cyclopropyl]amino]-5-(propylthio)-3H-1,2,3-triazolo[4,5-d]pyrimidin-3-yl]-1,2,3-cyclopentanetriol also called Fluometacyl or Fluometacyl® or a combination thereof.

22. The method according to claim anyone of claims 16 to 20 wherein bioactive molecule, therapeutic molecule or drug is a pyrimidine derivative represented by formula (4) 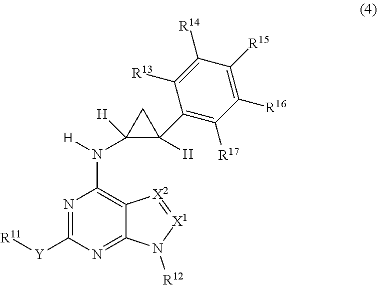
or optical isomers, racemic mixtures thereof, pharmaceutically acceptable acid addition salts, pharmaceutically acceptable metal salts, or alkylated ammonium salts or prodrug thereof;
wherein:
X¹ and X² are independently N, CH, CR⁸ wherein R⁸ is C₁₋₆ alkyl, C₂₋₆ alkenyl or C₂₋₆ alkynyl; with the exception that if one of X¹ or X² is equal to N, then the remaining X¹ or X² are selected from CH, CR⁸.
—Y— is —O— or —S—;
R¹¹ and R¹² are independently C₁₋₆-alkyl, C₂₋₆-alkenyl, C₂₋₆-alkynyl, C₃₋₆-cycloalkyl, aryl, aryl-C₁₋₆-alkyl wherein the alkyl or cycloalkyl moiety is optionally mono or polysubstituted with OH or an halogen and the aryl moiety is optionally mono or polysubstituted with an halogen, —C₁₋₆alkyl, —C₁₋₆alkoxy, —OH, —NO₂, —CN, —NH₂, —NHR⁸, —N(R⁸)₂—COOH, —COOR⁸, —CONH₂, —CONHR⁸, —CON(R⁸)₂, —SO₂NH₂, —SO₂NHR⁸, or —SO₂N(R⁸)₂;
R¹³, R¹⁴, R¹⁵, R¹⁶ and R¹⁷ are independently H, an halogen, a C₁₋₆alkyl, C₁₋₆alkoxy, —OH, —NO₂, —CN, —NH₂, —NHR⁸, —N(R⁸)₂—COOH, —COOR⁸, —CONH₂, —CONHR⁸, —CON(R⁸)₂, —SO₂NH₂, —SO₂NHR⁸, or —SO₂N(R⁸)₂.

23. The method according to any one of claims 16 to 20 wherein the one or more bioactive molecule, therapeutic molecule or drug are an antivirulence or a bactericidal agent together with a bacteriostatic agent, preferably minocycline or chlorhexidine.

24. A method of producing a medical device, a biomaterial implant or a bioprosthesis with a nanogel coated surface comprising one or more bioactive molecule, therapeutic molecule or drug together with an amphiphilic molecule; comprising the following-in-sequence steps of:
ci) optionally dipping the surface to be coated in a buffer solution of dopamine;
cii) dipping the surface coated at step ci) into a solution of polymer bearing primary or secondary amine groups; then
ciii) dipping the resulting coated surface obtained at step cii) into a liquid suspension of crosslinked-nanogel comprising one or more bioactive molecule, therapeutic molecule or drug together with the amphiphilic molecule; then
civ) drying the crosslinked coated surface obtained at step (ciii) to obtain a coated crosslinked-nanogel surface comprising one or more bioactive molecule, therapeutic molecule or drug together with the amphiphilic molecule;
cv) optionally repeating steps cii) to civ) to obtain a surface coated with several layers of crosslinked nanogels comprising one or more bioactive molecule, therapeutic molecule or drug together with the amphiphilic molecule.

25. A method of producing a medical device, a biomaterial implant or a bioprosthesis with a coated surface, wherein the coating is obtained by the following in-sequence steps:
i) optionally dipping the surface to be coated in a buffer solution of dopamine;
ii) dipping the surface coated with dopamine at step i); in a solution of polymer bearing primary or secondary amine groups; then
iii) dipping the resulting coated surface obtained at step ii) in a mixture of a poly(methacrylamide)-bearing quinone groups of formula (1) 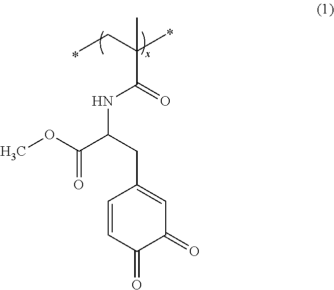
wherein x is an integer >1, preferably x is between 1 and 100
and/or of a poly(vinylquinone) of formula (6) 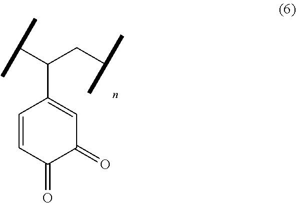
wherein n is an integer >1, preferably n is between 1 and 100;
with one or more bioactive molecule, therapeutic molecule or drug together with an amphiphilic molecule;
iv) drying the crosslinked coated surface obtained at step iii) to obtain a coated crosslinked monolayer surface comprising one or more bioactive molecule, therapeutic molecule or drug together with the amphiphilic molecule;
v) optionally repeating steps ii) to iv) to obtain a surface coated with several layers of crosslinked multilayers comprising one or more bioactive molecule, therapeutic molecule or drug together with the amphiphilic molecule, preferably the vitamin-E derivative.

26. The method according to claim 24 or 25 wherein the amphiphilic molecule is a vitamin-E derivative, preferably D-α-tocophenyl polyethylene glycol succinate.

27. The method according to claim 24 or 25 wherein the amphiphilic molecule is an amphiphilic cyclodextrine, preferably hydroxypropyl-beta-cyclodextrine.

28. The method according to any one of claims 224 to 27 wherein the one or more bioactive molecule, therapeutic molecule or drug is a triazolo(4,4-d)-pyrimidine derivative or a pyrimidine derivative together with a bacteriostatic agent, preferably minocycline or chlorhexidine.

29. The method according to anyone of claims 24 to 27 wherein the one or more bioactive molecule, therapeutic molecule or drugs is (1S,2S,3R,5S)-3-[7-[(1R,2S)-2-(3,4-difluorophenyl)cyclopropylamino]-5-(propylthio)-3H-[1,2,3]-triazolo[4,5-d]pyrimidin-3-yl]-5-(2-hydroxyethoxy)-1,2-cyclopentanediol) also called Triafluocyl; or (1S,2R,3S,4R)-4-[7-[[(1R,2S)-2-(3,4-Difluorophenyl)-cyclopropyl]amino]-5-(propylthio)-3H-1,2,3-triazolo[4,5-d]pyrimidin-3-yl]-1,2,3-cyclopentanetriol also called Fluometacyl or Fluometacyl®, or a combination thereof.

30. A pharmaceutical composition comprising a nanogel according to any one of claims 1 to 11 for use in treatment or prevention of bacterial virulence in a host mammal by topical administration.

31. Use of a nanogel according to any one of claims 1 to 11 as inhibitor of bacterial adhesion on a surface of the biomaterial implant, medical device or bioprothesis.

32. Use according to claim 31 wherein the biomaterial implant, medical device or bioprothesis is a catheter.
